SEQUENCE LISTING (6 sequences)

SEQ ID NO: 1 - protein, 226 aa, Escherichia coli
MNPIVINRLQRKLGYTFNHQELLQQALTHRSASSKHNERLEFLGDSILSYVIANALYHRFPRVDEGDMSRMRATLVRGNTLAELAREFELGECLRLGPGELKSGGFRRESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 2 - DNA, 355 bp, Artificial Sequence
  Description of Artificial Sequence  Synthetic Primer
tacgatttaggtgacactatagaatacacggaattaatacgactcactatagggaattaccctcactaaagggaggaagctgcaattgggatgcaagctttccacatctggcacagagcgggaggtctctgagccactgggcctagatgatgccggaaacaagaagtcatcaaagggttctgccttcagctccacgttgctgatgctcttgactggctccaaggatggcttgggctcagggtcgttgagaaggggcagggtgaaggcctcctcagactctggggtggaagcctcaggcagacctccagtcaaatccagggaggccacagacatctcctctgggaagccaagaatt

SEQ ID NO: 3 - DNA, 40 bp, Artificial Sequence
  Description of Artificial Sequence  Synthetic Primer
aatttaatacgactcactataggaagcattgagcaaatcc

SEQ ID NO: 4 - DNA, 41 bp, Artificial Sequence
  Description of Artificial Sequence  Synthetic Primer
aatttaatacgactcactataggcttctggccaggggtctc

SEQ ID NO: 5 - DNA, 41 bp, Artificial Sequence
  Description of Artificial Sequence  Synthetic Primer
aatttaatacgactcactataggtaccagaagcggtgccgg

SEQ ID NO: 6 - DNA, 43 bp, Artificial Sequence
  Description of Artificial Sequence  Synthetic Primer
aatttaatacgactcactataggcaaacgactgtcctggccgt

(19) United States
(12) Patent Application Publication
(10) Pub. No.: US 2012/0028312 A1
(43) Pub. Date: Feb. 2, 2012

(54) METHODS AND COMPOSITIONS RELATING TO POLYPEPTIDES WITH RNASE III DOMAINS THAT MEDIATE RNA INTERFERENCE

(75) Inventors: Lance P. Ford, Austin, TX (US); David Brown, Austin, TX (US)

(73) Assignee: LIFE TECHNOLOGIES CORPORATION, Carlsbad, CA (US)

(21) Appl. No.: 13/196,710

(22) Filed: Aug. 2, 2011

Related U.S. Application Data
(63) Continuation of application No. 12/559,276, filed on Sep. 14, 2009.
Application No. 12/559,276 is a continuation of application No. 10/460,775, filed on Jun. 12, 2003, now abandoned.
Application No. 10/460,775 is a continuation-in-part of application No. 10/360,772, filed on Jun. 12, 2002, now abandoned.
(60) Provisional application No. 60/402,347, filed on Aug. 10, 2002.

(51) Int. Cl. C12P 19/34; C12N 9/12; C12N 9/22

(52) U.S. Cl. 435/91.3; 435/199; 435/194

(57) ABSTRACT

The present invention concerns methods and compositions involving RNase III and polypeptides containing RNase III domains to generate RNA capable of triggering RNA-mediated interference (RNAi) in a cell. In some embodiments, the RNase III is from a prokaryote. RNase III activity will cleave a double-stranded RNA molecule into short RNA molecules that may trigger or mediate RNAi (siRNA). Compositions of the invention include kits that include an RNase III domain-containing polypeptide. The present invention further concerns methods using polypeptides with RNase III activity for generating RNA molecules that effect RNAi, including the generation of a number of RNA molecules to the same target.

This application is a continuation of U.S. patent application Ser. No. 12/559,276 filed Sep. 14, 2009, which application is a continuation of U.S. patent application Ser. No. 10/460,775 filed on Jun. 12, 2003 which application claims the benefit of U.S. Provisional Patent Application No. 60/402,347 filed Aug. 10, 2002 and claims priority to U.S. patent application Ser. No. 10/360,772 filed on Jun. 12, 2002 (formerly 60/388,547), all of which are hereby incorporated by reference in their entirety.

### BACKGROUND OF THE INVENTION

1. Field of the Invention

The present invention relates generally to the field of molecular biology. More particularly, it concerns RNase III and polypeptides with an RNase III domain and the use of such proteins to generate multiple double-stranded RNA, as well as pools of dsRNA, capable of reducing target gene expression in vitro and in vivo.

2. Description of the Related Art

RNA interference (RNAi), originally discovered in Caenorhabditis elegans by Fire and Mello (Fire et al., 1998), is a phenomenon in which double stranded RNA (dsRNA) reduces the expression of the gene to which the dsRNA corresponds. The phenomenon of RNAi was subsequently proven to exist in many organisms and to be a naturally occurring cellular process. The RNAi pathway can be used by the organism to inhibit viral infections, transposon jumping and to regulate the expression of endogenous genes (Huntvagner et al., 2001; Tuschl, 2001; Waterhouse et al., 2001; Zamore 2001). In original studies, researchers were inducing RNAi in non-mammalian systems and were using long double stranded RNAs. However, most mammalian cells have a potent antiviral response causing global changes in gene expression patterns in response to long dsRNA thus arousing questions as to the existence of RNAi in humans. As more information about the mechanistic aspects of RNAi was gathered, RNAi in mammalian cells was shown to also exist.

In an in vitro system derived from Drosophila embryos long dsRNAs are processed into shorter small interfering (si) RNA the smaller siRNA by a cellular ribonuclease containing RNaseIII motifs (Bernstein et al., 2001; Grishok et al., 2001; Hamilton and Baulcombe, 1999; Knight and Bass, 2001; Zamore et al., 2000). Genetics studies done in C. elegans, N. crassa and A. thaliana have lead to the identification of additional components of the RNAi pathway. These genes include putative nucleases (Ketting et al., 1999), RNA-dependent RNA polymerases (Cogoni and Macino, 1999a; Dalmay et al., 2000; Mourrain et al., 2000; Smardon et al., 2000) and helicases (Cogoni and Macino, 1999b; Dalmay et al., 2001; Wu-Scharf et al., 2000). Several of these genes found in these functional screens are involved not only in RNAi but also in nonsense mediated mRNA decay, protection against transposon-transposition (Zamore, 2001), viral infection (Waterhouse et al., 2001), and embryonic development (Hutvagner et al., 2001; Knight and Bass, 2001). In general, it is thought that once the siRNAs are generated from longer dsRNAs in the cell by the RNaseIII like enzyme, the siRNA associate with a protein complex. The protein complex also called RNA-induced silencing complex (RISC), then guides the smaller 21 base double stranded siRNA to the mRNA where the two strands of the double stranded RNA separate, the antisense strand associates with the mRNA and a nuclease cleaves the mRNA at the site where the antisense strand of the siRNA binds (Hammond et al., 2001). The mRNA is then subsequently degraded by cellular nucleases.

Based upon some of the information mentioned above, Elbashir et al. (2001) discovered a clever method to bypass the anti viral response and induce gene specific silencing in mammalian cells. Several 21 nucleotide dsRNAs with 2 nucleotide 3′ overhangs were transfected into mammalian cells without inducing the antiviral response. The small dsRNA molecules (also referred to as “siRNA”) were capable of inducing the specific suppression of target genes. In one set of experiments, siRNAs complementary to the luciferase gene were co-transfected with a luciferase reporter plasmid into NIH3T3, COS-7, HeLaS3, and 293 cells. In all cases, the siRNAs were able to specifically reduce luciferase gene expression. In addition, the authors demonstrated that siRNAs could reduce the expression of several endogenous genes in human cells. The endogenous targets were lamin A/C, lamin B1, nuclear mitotic apparatus protein, and vimentin. The use of siRNAs to modulate gene expression has now been reproduced by at least two other labs (Caplen et al., 2001; Hutvagner et al., 2001) and has been shown to exist in more that 10 different organisms spanning a large spectrum of the evolutionary tree. RNAi in mammalian cells has the ability to rapidly expand our knowledge of gene function and cure and diagnose human diseases. However, much about the process is still unknown and thus, additional research and understanding will be required to take full advantage of it.

The making of siRNAs has been through direct chemical synthesis, through processing of longer double stranded RNAs exposure to Drosophila embryo lysates, through an in vitro system derived from S2 cells, using page polymerase promoters, RNA-dependant RNA polymeras, and DNA based vectors. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA.

WO 99/32619 and WO 01/68836 suggest that RNA for use in siRNA may be chemically or enzymatically synthesized. The enzymatic synthesis contemplated is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914 suggests that single strands of RNA can be produced enzymatically or by partial/total organic synthesis. Preferably, single stranded RNA is enzymatically synthesized from the PCR products of a DNA template, preferably a cloned cDNA template and the RNA product is a complete transcript of the cDNA, which may comprise hundreds of nucleotides. WO 01/36646 places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences. U.S. Pat. No. 5,795,715 was filed Jun. 17, 1994, well before the phenomenon of RNA interference was described by Fire, et al. (1998). The production of siRNA was therefore, not contemplated by these authors.

In the provisional patent 60/353,332, which is specifically incorporated by reference, the production of siRNA using the RNA dependent RNA polymerase, P2 and that this dsRNA can be used to induce gene silencing. Although this method is not commercially available or published in a scientific journal it was determined to be feasible. Several laboratories have demonstrated that DNA expression vectors containing mammalian RNA polymerase III promoters can drive the expression of siRNA that can induce gene-silencing (Brummelkamp et al., 2002; Sui et al., 2002; Lee et al., 2002; Yu et al., 2002; Miyagishi et al., 2002; Paul et al., 2002). The RNA produced from the polymerase III promoter can be designed such that it forms a predicted hairpin with a 19-base stem and a 3-8 base loop. The approximately 45 base long siRNA expressed as a single transcription unit folds back on it self to form the hairpin structure as described above. Hairpin RNA can enter the RNAi pathway and induce gene silencing. The siRNA mammalian expression vectors have also been used to express the sense and antisense strands of the siRNA under separate polymerase III promoters. In this case, the sense and antisense strands must hybridize in the cell following their transcription (Lee et al., 2002; Miyagishi et al., 2002). The siRNA produced from the mammalian expression vectors weather a hairpin or as separate sense and antisense strands were able to induce RNAi without inducing the antiviral response. More recent work described the use of the mammalian expression vectors to express siRNA that inhibit viral infection (Jacque et al., 2002; Lee et al., 2002; Novina et al., 2002). A single point mutation in the siRNA with respect to the target prevents the inhibition of viral infection that is observed with the wild type siRNA. This suggests that siRNA mammalian expression vectors and siRNA could be used to treat viral diseases.

An alternative enzymatic approach to siRNA production that elevates the need to perform screens for siRNA that are functional. Currently, a 4 or more siRNA to one target need to be designed to a single target. A siRNA synthesis method that would get around transfecting 4 or more separate siRNA per target would be beneficial in cost and time. Therefore, a method in which a mixture of siRNA can be made from a single reaction would increase the likely hood of knocking down the gene the first time it is performed. In order to generate this mixture of siRNA one approach would be using RNaseIII type nucleases could be used. Recombinant bacterial RNaseIII (25.6 KDa) is one such nuclease that can cleave long dsRNA into short dsRNAs containing a 5′-PO₄ and a 2 nucleotide 3′ overhang. Although the RNA cleaved by bacterial RNaseIII are generally smaller (12-15 bases in length) it leaves a 5′PO₄ and a 2-nucleotide 3′ overhang which is the same structure found on the RNA produced by DICER. A second approach would be to produce a mixture of siRNA and transfecting in the mixture of siRNA into the same reaction. The siRNA can be generated using a number of approaches currently methods for siRNA production-include chemical synthesis, in vitro synthesis using phase polymerase promters, RNA dependant RNA polymerase or DNA vector based approaches.

RNase III is conserved in all known bacteria and eukaryotes and has 1-2 copies of a 9-residue consensus sequence, known as the RNase III signature motif. The bacterial RNase III proteins are the simplest, consisting of two domains: an N-terminal endonuclease domain, followed by a double-stranded RNA binding domain (dsRBD) (Blaszczyk et al, 2001). As described, the RNase III protein consists of two modules, a approximately 150 residue N-terminal catalytic domain and a approximately 70 residue C-terminal recognition module, homologous with other dsRBDs. While forms of RnaseIII can act as dimers others are able to act as monomers. For example, the more complex versions of RNaseIII domain-containing proteins such as DICER contain two domes of the RNaseIII motif, dsRNA binding domain, and a DEAH RNA helicase domain and a PAZ domain and is believed to function as a monomer. The structure of the approximately 70 residue dsRNA binding domain of bacterial RNaseIII was identified (Kharrat et al, 1995).

Dicer is a eukaryotic protein that cleaves double-stranded RNA into 21-25 siRNA (Bernstein et al., 2001; Elbashir et al., 2001). The use of Dicer for in vitro generation of siRNA is problematic, however, because the reaction can be inefficient (Bernstein et al., 2001) and it is difficult to purify for in vitro application.

Not all small, double-stranded RNA molecules can effect RNA interference of a target gene. Such molecules require assaying to determine whether they possess this activity, which can be time consuming. Thus, it would be advantageous to be able to generate a pool of small, double-stranded RNA molecules, one or more of which may mediate RNA interference. Employing a pool of candidate dsRNA molecules could avoid the need to assay which molecules work and which do not. Thus, there is a need for the ability to generate and use such pools of small, dsRNA to implement RNAi.

### SUMMARY OF THE INVENTION

The present invention is based on the inventors' discovery that RNase III can generate one or more double stranded ribonucleic acid molecules capable of reducing the expression of a targeted gene through RNAi (referred to as “dsRNA” or “siRNA”). Thus, the present invention is directed to compositions and methods involving polypeptides that contain an RNase III domain to generate small, double-stranded RNA molecules that effect, trigger, or induce RNAi (termed “siRNA molecules,” which refers to RNA molecules that have a least one double stranded region and the ability to effect RNAi). RNAi is mediated by an RNA-induced silencing complex (RISC), which associates (specifically binds one or more RISC components) with dsRNA of the invention and guides the dsRNA to its target mRNA through base-pairing interactions. Once the dsRNA is base-paired with its mRNA target, nucleases cleave the mRNA.

In some embodiments, the invention concerns a dsRNA or siRNA that is capable of triggering RNA interference, a process by which a particular RNA sequence is destroyed. siRNA are dsRNA molecules that are 100 bases or fewer in length (or have 100 basepairs or fewer in its complementarity region). In some cases, it has a 2 nucleotide 3′ overhang and a 5′ phosphate. The particular RNA sequence is targeted as a result of the complementarity between the dsRNA and the particular RNA sequence. It will be understood that dsRNA or siRNA of the invention can effect at least a 20, 30, 40, 50, 60, 70, 80, 90 percent or more reduction of expression of a targeted RNA in a cell. dsRNA of the invention (the term “dsRNA” will be understood to include “siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. The complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases. In some embodiments, long dsRNA are employed in which “long” refers to dsRNA that are 1000 bases or longer (or 1000 basepairs or longer in complementarity region). The term “dsRNA” includes “long dsRNA” and “intermediate dsRNA” unless otherwise indicated. In some embodiments of the invention, dsRNA can exclude the use of siRNA, long dsRNA, and/or “intermediate” dsRNA (lengths of 100 to 1000 bases or basepairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (see Sui et al., 2002 and Brummelkamp et al., 2002 in which a single strand with a hairpin loop is used as a dsRNA for RNAi). For convenience, lengths of dsRNA may be referred to in terms of bases, which simply refers to the length of a single strand or in terms of basepairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In the range of 50% to 70% complementarity, such sequences may be referred to as “very complementary,” while the range of greater than 70% to less than complete complementarity can be referred to as “highly complementary.” Unless otherwise specified, sequences that are “complementary” include sequences that are “very complementary,” “highly complementary,” and “fully complementary.” It is also contemplated that any embodiment discussed herein with respect to “complementary” strands or region can be employed with specifically “fully complementary,” “highly complementary,” and/or “very complementary” strands or regions, and vice versa. Thus, it is contemplated that in some instances, as demonstrated in the Examples, that siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability.

It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complentarity region.

The single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-length of a particular gene's mRNA without the poly-A tail) bases or basepairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or basepairs) or less, in which case they may also be referred to as “siRNA.” In specific embodiments the strand or strands of the dsRNA are less than 70 bases in length. With respect to those embodiments, the dsRNA strand or strands may be from 5-70, 10-65, 20-60, 30-55, 40-50 bases or basepairs in length. A dsRNA that has a complementarity region equal to or less than 30 basepairs (such as a single stranded hairpin RNA in which the stem or complementary portion is less than or equal to 30 basepairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 basepairs or fewer. In some cases, a dsRNA may be processed in the cell into siRNA.

The present invention is based on the discovery that prokaryotic RNase III can be used to generate siRNA molecules from double-stranded RNA. Thus, the present invention concerns compositions and methods involving RNase III to generate siRNA to effect RNA interference in a cell. The term “siRNA” refers to an RNA molecule that has at least one double stranded region and that can reduce, inhibit, or eliminate the expression of a target gene in a cell, which is a process known as RNA interference or RNA-mediated interference.

Methods and compositions, including kits, of the invention concern RNase III, which is an enzyme that cleaves double stranded RNA into one or more pieces that are 12-30 base pairs in length, or 12-15 basepairs or 20-23 basepairs in length in some embodiments Thus, candidate siRNA molecules (which refers to dsRNA that are the appropriate length to mediate or trigger RNAi, but it is not yet known whether it can achieve RNAi) may be 12, 13, 14, 15, 16, 17, 18, 19, 20, 21, 22, 23, 24, 25, 26, 27, 28, 29, or 30 basepairs in length.

It is specifically contemplated that the eukaryotic protein Dicer is excluded as part of the invention in some embodiments. In further embodiments of the invention, RNase III is from a prokaryote, including a gram negative bacteria. Thus, the present invention may refer to a “non-eukaryotic RNase III” to exclude eukaryotic-derived proteins such as Dicer or it may refer to “prokaryotic RNase III” to refer to an RNase III protein derived from a prokaryotic organism. In additional embodiments of the invention, the RNase III is from E. coli, a gram-negative bacteria. The RNase III from E. coli may have the amino acid sequence of GenBank Accession Number NP_{—}289124 (SEQ ID NO:1), which is specifically incorporated by reference.

In further embodiments of the invention, methods and compositions involve a protein or polypeptide with RNase III activity (that is, the ability to cleave double stranded RNA into smaller segments) or a protein or polypeptide with an RNase III domain. An “RNase III domain” refers to an amino acid region that confers the ability to cleave double stranded RNA into smaller segments, and which is understood by those of skill in the art and as described elsewhere herein.

In other compositions and methods of the invention, the RNase III may be purified from an organism's endogenous supply of RNase III; alternatively, recombinant RNase III may be purified from a cell or an in vitro expression system. The term “recombinant” refers to a compound that is produced by from a nucleic acid (or a replicated version thereof) that has been manipulated in vitro, for example, being digested with a restriction endonuclease, cloned into a vector, amplified, etc. The terms “recombinant RNase III” and “recombinantly produced RNase III” refer to an active RNase III polypeptide that was prepared from a nucleic acid that was manipulated in vitro or is the replicated version of such a nucleic acid. It is specifically contemplated that RNase III may be recombinantly produced in a prokaryotic or eukaryotic cell. It may be produced in a mammalian cell, a bacterial cell, a yeast cell, or an insect cell. In specific embodiments of the invention, the RNase III is produced from a baculovirus expression system involving insect cells. Alternatively, recombinant RNase III may be produced in vitro or it may be chemically synthesized. Such RNase III may first be purified for use in RNA interference. Purification may allow the RNAse III to retain activity in concentrations of about 0.1, 0.2, 0.3, 0.4, 0.5, 0.6, 0.7, 0.8, 0.9, 1, 2, 3, 4, 5, 6, 7, 8, 9, 10 or more units/microliter. A “unit” is defined as the amount of enzyme that digests 1 μg of a 500 basepair dsRNA in 60 minutes at 37° C. into RNA products that are 12-15 basepairs in length.

It is contemplated that the use of the term “about” in the context of the present invention is to connote inherent problems with precise measurement of a specific element, characteristic, or other trait. Thus, the term “about,” as used herein in the context of the claimed invention, simply refers to an amount or measurement that takes into account single or collective calibration and other standardized errors generally associated with determining that amount or measurement. For example, a concentration of “about” 100 mM of Tris can encompass an amount of 100 mM±5 mM, if 5 mM represents the collective error bars in arriving at that concentration. Thus, any measurement or amount referred to in this application can be used with the term “about” if that measurement or amount is susceptible to errors associated with calibration or measuring equipment, such as a scale, pipetteman, pipette, graduated cylinder, etc.

RNase III polypeptides or polypeptides with an RNase III domain or activity may be used in conjunction with an enzyme dilution buffer. In some embodiments, the composition comprises an enzyme dilution buffer. The enzymes of the invention may be provided in such a buffer. In some embodiments, the buffer comprises one or more of the following glycerol, Tris, dithiothreitol (DTT), or EDTA. In specific embodiments, the enzyme dilution buffer comprises 50% glycerol, 20 mM Tris, 0.5 mM DTT, and 0.5 mM EDTA. In a method employing a composition, these components of the buffer may be diluted after addition of other components to the composition.

In still further embodiments of the invention, recombinantly produced RNase III may be truncated by or be missing 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids in one or more places in the polypeptide, yet still retain RNase III activity. In addition or alternatively, an RNase III polypeptide may include a heterologous sequence of at least 3 amino acids and also still retain RNase III activity. The heterologous sequence may be a discernible region (contiguous stretch of amino acids) from another polypeptide to render the RNase III polypeptide chimeric. The heterologous sequence may be tag that facilitates production or purification of the RNase III. Thus, in some embodiments of the invention, recombinant RNase III has a tag attached to it, either on one of its ends or atached at any residue in between. In some embodiments the tag is a histidine tag (His-tag), which is a series of at least 3 histidine residues and in some embodiments, 4, 5, 6, 7, 8, 9, 10, or more consecutive histidine residues. In other embodiments, the tag is GST, streptavidin, or FLAG. Additionally, some RNase III polypeptides may have a tag initially, but the tag may be removed subsequently.

Furthermore, it is contemplated that siRNA or the longer dsRNA template may be labeled. The label may be fluorescent, radioactive, enzymatic, or colorimetric.

The substrate for RNase III of the invention is a dsRNA molecule, which may be composed of two strands or a single strand with a region of complementarity within the strand. It is contemplated that the dsRNA substrate may be 25 to 10,000, 25 to 5,000, 50 to 1,000, 100-500, or 100-200 nucleotides or basepairs in length. Alternatively the dsRNA substrate may be at least or at most 25, 50, 75, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, 6000, 6500, 7000, 7500, 8000, 8500, 9000, 9500, or 10,000 or more nucleotides of basepairs in length. dsRNA need only correspond to part of the target gene to yield an appropriate siRNA. Thus, a dsRNA that corresponds to all or part of a target gene means that the dsRNA can be cleaved to yield at least one siRNA that can silence the target gene. The dsRNA may contain sequences that do not correspond to the target gene, or the dsRNA may contain sequences that correspond to multiple target genes.

The invention also concerns labeled dsRNA. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C).

When two or more differentially colored labels are employed, fluorescent resonance energy transfer (FRET) techniques may be employed to characterize the dsRNA.

Labels contemplated for use in several embodiments are non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof. The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation.

Any cell that undergoes RNAi can be employed in methods of the invention. The cell may be a eukaryotic cell, mammalian cell such as a primate, rodent, rabbit, or human cell, a prokaryotic cell, or a plant cell. In some embodiments, the cell is alive, while in others the cell or cells is in an organism or tissue. Alternatively, the cell may be dead. The dead cell may also be fixed. In some cases, the cell is attached to a solid, non-reactive support such as a plate or petri dish. Such cells may be used for array analysis. It is contemplated that cells may be grown on an array and dsRNA administered to the cells.

In some embodiments of the invention, there are methods of reducing the expression of a target gene in a cell. Such methods involve the compositions described above, including the embodiments described for RNase III, dsRNA, and siRNA.

Methods of the reducing expression of a target gene involve a) incubating a dsRNA corresponding to part of the target gene with an effective amount of composition comprising RNase III under conditions to allow RNase III to cleave the dsRNA into siRNA; and b) introducing the siRNA into the cell. The term “effective amount” in the context of RNase III refers to an amount that will effect cleavage of a dsRNA substrate by RNase III. “Target gene” or “targeted gene” refers to a gene whose expression is desired to be reduced, inhibited or eliminated through RNA interference. RNA interference directed to a target gene requires an siRNA that is complementary in one strand and identical in the other strand to a portion of the coding region of the targeted gene. siRNA may be introduced into a cell by transfection or infection. Such techniques are well known to those of skill in the art and include, but are not limited to, the use of calcium phosphate, liposomes such as lipofectamine, electroporation, and plasmids and vectors including viral vectors.

In additional methods of the invention, a dsRNA may be the substrate for RNase III activity, but only some of the resulting products are characterized as siRNA because not all of the products can effect RNAi. The products of dsRNA cleavage by RNase III are candidate siRNAs. By processing a long dsRNA, the need for determining which RNA product is an siRNA is rendered moot or diminished.

Further embodiments of the invention concern generating candidate siRNA to trigger RNAi in a cell to a target gene. Any of the methods described herein for reducing the expression of a target gene can be applied to generating candidate siRNA and vice versa. Furthermore, it is specifically contemplated that the generation of candidate siRNA from a longer dsRNA molecule may be done outside of a cell (in vitro). In fact, particular embodiments of the invention take advantage of the benefits of employing compositions that can be manipulated in a test tube, as opposed to in a cell.

In additional methods of the invention at least one siRNA molecule is isolated away from the other siRNA molecules. However, it is specifically contemplated that all or a subset of the candidate siRNA products that result from RNase III cleavage(s) may be employed in methods of the invention. Thus, pools of candidate siRNAs directed to a single or multiple targets may be transfected or administered to a cell to trigger RNAi against the target(s).

In some methods of the invention, siRNA molecules or template nucleic acids may be isolated or purified prior to their being used in a subsequent step. SiRNA molecules may be isolated or purified prior to transfection into a cell. A template nucleic acid or amplification primer may be isolated or purified prior to it being transcribed or amplified. Isolation or purification can be performed by a number of methods known to those of skill in the art with respect to nucleic acids. In some embodiments, a gel, such as an agarose or acrylamide gel, is employed to isolate the siRNA.

In some methods of the invention dsRNA is obtained by transcribing each strand of the dsRNA from one or more cDNA (or DNA or RNA) encoding the strands in vitro. It is contemplated that a single template nucleic acid molecule may be used to transcribe a single RNA strand that has at least one region of complementarity (and is thus double-stranded under conditions of hybridization) or it may be used to transcribe two separate complementary RNA molecules. Alternatively, more than one template nucleic acid molecule may be transcribed to generate two separate RNA strands that are complementary to one another and capable of forming a dsRNA.

Additional methods involve isolating the transcribed strand(s) and/or incubating the strand(s) under conditions that allow the strand(s) to hybridize to their complementary strands (or regions if a single strand is employed).

Nucleic acid templates may be generated by a number of methods well known to those of skill in the art. In some embodiments the template, such as a cDNA, is synthesized through amplification or it may be a nucleic acid segment in or from a plasmid that harbors the template.

Other methods of the invention also concern transcribing a strand or strands of a dsRNA using a promoter that can be employed in vitro or outside a cell, such as a prokaryotic promoter. In some embodiments, the prokaryotic promoter is a bacterial promoter or a bacteriophage promoter. It is specifically contemplated that dsRNA strands are transcribed with SP6, T3, or T7 polymerase.

Methods for generating siRNA to more than one target gene are considered part of the invention. Thus, siRNA or candidate siRNA directed to 1, 2, 3, 4, 5, 6, 7, 8, 9, 10 or more target genes may be generated and implemented in methods of the invention. An array can be created with pools of siRNA to multiple targets may be used as part of the invention.

In specific embodiments of the invention, there are methods for achieving RNA interference of a target gene in a cell using one or more siRNA molecules. These methods involve: a) generating at least one double-stranded DNA template (which may comprise an SP6, T3, or T7 promoter on at least one strand) corresponding to part of the target gene; b) transcribing the template, wherein either i) a single RNA strand with a complementarity region is created or ii) first and second complementary RNA strands are created; c) hybridizing either the single complementary RNA strand or the first and second complementary RNA strands to create a dsRNA molecule corresponding to the target gene; d) incubating the dsRNA molecule with a polypeptide comprising an RNase III domain, under conditions to allow cleavage of the dsRNA into at least two candidate siRNA molecules; and, e) transfecting at least one siRNA into the cell.

In some methods of the invention, a candidate siRNA may be tested for its ability to mediate or trigger RNAi, however, in some embodiments of the invention, it is not assayed. Instead, multiple siRNAs directed to different portions of the same target may be employed to reduce expression of the target.

It is specifically contemplated that any method of the invention may be employed with any kit component or composition described herein. Furthermore, any kit may contain any component described herein and any component involved in any method of the invention. Thus, any element discussed with respect to one embodiment may be applied to any other embodiment of the invention.

The present invention concerns kits that can be used to generate siRNA and siRNA candidate molecules. Components of the kit may be provided in concentrations of about 1×, 2×, 3×, 4×, 5×, 6×, 7×, 8×, 9×, 10×, 15×, 20×, 25× or higher with respect to final reaction volumes. Such concentrations apply specifically with respect to buffers in the kit.

In kit embodiments, kits include a) recombinant, prokaryotic RNase III; b) RNase III buffer; and, c) a control nucleic acid. The RNase III may be provided in an enzyme dilution buffer.

Kits may also include an RNase III buffer. The RNase III enzymes of the invention may be used with an RNase III buffer. Such a buffer facilitates enzyme activity. In some embodiments of the invention, the RNase buffer comprises Tris and a salt. In specific embodiments, the salt is NaCl, MgCl₂, or CaCl₂. In other embodiments, the buffer comprises MgCl₂ and CaCl₂. The buffer may be at a concentration of 2× to 20×. In certain embodiments the RNase III buffer is 5×. The 5× RNase III buffer comprises about 50 mM Tris, about 0.5 mM CaCl₂, about 12.5 mM MgCl₂, and about 800 mM NaCl. In other embodiments, the RNase III buffer is 10× concetration and comprises about 100 mM Tris, about 1 mM CaCl₂, and about 25 mM MgCl₂.

Kits of the invention may also comprise one or more of the following 1) SP6, T3 or T7 RNA polymerase; 2) a SP6, T3 or T7 RNA polymerase buffer; 3) NTPs or dNTPs; 4) RNase A; 5) RNase buffer; 6) RNase and/or DNase inhibitor; and/or 7) control nucleic acid.

Several kit components comprise Tris or Tris-HCl. It may have a pH in the range of about 6.5 to 8.5, though in many embodiments the pH is about 7.0, 7.5, or 8.0. Also, it is provided at a concentration of about 50 mM, 100 mM, 150 mM, 200 mM or higher in many embodiments.

In some embodiments, RNA polymerase is provided as a concentration of about 100 units/ml. Th polymerase may be in an enzyme mix comprising inorganic pyrophosphatase, at least one RNase inhibitor, and about 1% CHAPS. In some embodiments, the enzyme mix comprises two RNase inhibitors. The concentration of inorganic pyrophosphatase is about 0.05 units/ml and the concentration of the RNase inhibitor is about 0.3 units/ml and about 2 units/ml in other embodiments. Furthermore, in other embodiments the enzyme mix comprises SUPERase.In™ RNase Inhibitor at a concentration of about 2 units/ml.

Polymerase buffers may be included in a kit or used with a method of the invention. In some embodiments, the buffer is provided at a concentration of 2× to 20×. The buffer is provided at a concentration of 10× in specific embodiments and comprises about 400 mM Tris, about 200-300 mM MgCl₂, about 20 mM Spermidine, and about 100 mM DTT.

The kit may also comprise NTPs or dNTPs. NTPs include ATP, CTP, GTP, and/or UTP. In certain embodiments, the concentration of ATP, CTP, GTP, and UTP is each about 10, 25, 50, 75, or 100 mM.

The control nucleic acid may be DNA or RNA. If it is DNA, in some embodiments it comprises an SP6, T3, or T7 promoter. In some embodiments, control nucleic acids are a DNA template that are capable of being transcribed into RNA. In other embodiments, the control nucleic acid is a dsRNA or one or more RNA strands than can be hybridized to create a dsRNA In specific embodiments, the control nucleic acid has a sequence corresponding to (identical or complementary sequences) GAPDH or c-myc or La.

RNase A can be employed in methods of the invention and/or as a kit component. The concentration of RNase A is about 1 mg/ml in some embodiments. RNase A digestion buffer is also included in some embodiments.

In additional embodiments, the RNase A digestion buffer comprises about 100 mM Tris, about 25 mM MgCl₂, and about 5 mM CaCl₂.

Methods and kits may also involve a cartridge, column, or filter for isolating or purifying nucleic acids. In some embodiments, these comprise glass fiber. In that context there may be a binding buffer. In some embodiments, the binding buffer is 2× to 20×. In specific embodiments, the binding buffer is 10×. A 10× binding buffer comprises 5 M NaCl. Additionally, there may be a wash buffer. The wash buffer may be 2× to 5×. In certain embodiments, the wash buffer is 2×, which, in further embodiments, comprises 1 M NaCl. After the nucleic acids are bound and then washed, they may be eluted using an elution solution. The elution solution, in some aspects of the invention, comprises Tris and EDTA. In additional embodiments, the Tris is at a concentration of 10 mM and the EDTA is at a concentration of 1 mM in the elution solution.

Other components of the kit may be included to reduce or eliminate contamination issues that would impair the ability to generate an siRNA that could trigger RNAi. Thus, in some embodiments of the invention, there is nuclease-free water or nuclease-free equipment, such as tips, tubes, or other containers.

Specific kit embodiments are contemplated. In some embodiments, a kit for generating siRNA molecules comprises: a) prokaryotic RNase III in an enzyme dilution buffer comprising about 50% glycerol, about 20 mM Tris, about 0.5 mM DTT, and 0.5 mM EDTA. In still further embodiments, this kit includes a nucleic acid control.

In still further embodiments, there is a kit for generating siRNA molecules comprising: a) T7 polymerase in an enzyme mix comprising inorganic pyrophosphatase and at least one RNAse inhibitor in about 1% CHAPS; b) T7 polymerase buffer at a 10× concentration comprising about 400 mM Tris, about 200-300 mM MgCl₂, about 20 mM Spermidine, and about 100 mM DTT; c) prokaryotic RNase III in an enzyme dilution buffer comprising about 50% glycerol, about 20 mM Tris, about 0.5 mM DTT, and 0.5 mM EDTA; d) RNase III 10× buffer comprising about 100 mM Tris, about 1 mM CaCl₂, and about 25 mM MgCl₂; e) a control nucleic acid. This kit may further comprise one or more (including all) of the following: f) an NTP mix comprising ATP, CTP, GTP, and UTP; g) RNase A; h) RNase A digestion buffer comprising about 100 mM Tris, about 25 mM MgCl₂, and about 5 mM CaCl₂; i) glass fiber filter cartridge; j) 10× binding buffer comprising about 5 M NaCl; k) 2× wash buffer comprising about 1 M NaCl; 1) elution solution comprising Tris and EDTA.

All methods of the invention may use kit embodiments to achieve a method of reducing the expression of a target gene in a cell or for simply generating an siRNA or a candidate siRNA.

The present invention also concerns kits for labeling and using dsRNA for RNA interference. Kits may comprise components, which may be individually packaged or placed in a container, such as a tube, bottle, vial, syringe, or other suitable container means. Kit embodiments include the one of more of the following components: labeling buffer comprising a physiological buffer with a pH range of 7.0 to 7.5; labeling reagent for labeling dsRNA with fluorescent label comprising an alkylating agent; control dsRNA comprising a dsRNA known to trigger RNAi in a cell, such as those disclosed herein, nuclease free water, ethanol, NaCl, reconstitution solution comprising DMSO or annealing buffer comprising Hepes and at least one salt. In further embodiments, the labeling reagent comprises Cy3, Cy5, and/or fluorescein (FAM).

The salt in the annealing buffer, in some embodiments, is potassium acetate and/or magnesium acetate. Annealing buffer may contain 1, 2, 3, 4, 5, 6, 7, 8, 9, 10, 15, 20, 25, 30, 35, 40, 45, 50, 100, 150, 200, 250, 300, 350, 400, 450, 500, 550, 600, 650, 700, 750, 800, 850, 900, 950, 1000, 2000, 3000, 4000, 5000 mM or more of a salt such as potassium acetate and/or magnesium acetate, and/or sodium acetate. It may also contain a buffer such as Hepes or Tris in a concentration of 10, 15, 20, 30, 40, 50, 60, 70, 80, 90, 100, 110, 120, 130, 140, 150, 160, 170, 180, 190, 200, 210, 220, 230, 240, 250 mM or more, with a pH in the range of 7.0-8.0. In one embodiment, a 5× concentration of annealing buffer comprises 150 mM Hepes, pH 7.4, 500 mM potassium acetate, and 10 mM magnesium acetate. Other concentrations may be adjusted accordingly. It is contemplated that kits may contain any component to create compositions of the invention and to implement methods of the invention.

Individual components may also be provided in a kit in concentrated amounts; in some embodiments, a component is provided individually in the same concentration as it would be in a solution with other components. Concentrations of components may be provided as 1×, 2×, 5×, 10×, 15×, or 20× or more.

Control dsRNA is included in some kit embodiments. Control dsRNA is dsRNA that can be used as a positive control for labeling and/or RNAi. The control may be provided as a single strand or as two strands.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The following drawings form part of the present specification and are included to further demonstrate certain aspects of the present invention. The invention may be better understood by reference to one or more of these drawings in combination with the detailed description of specific embodiments presented herein.

FIG. 1A-B. Gels showing purification of bacterial RNase III and its short dsRNA products. A. Protein gel showing purification of bacterial RNase III using a nickel column. The arrow indicates the RNase III protein with the expected size of 30 kD. B. An acrylamide gel showing the RNA products produced by incubating the purified RNase III with dsRNA substrate.

FIG. 2A-C. Gels showing the generation of small dsRNA for siRNA directed to the human La gene product. A. Increasing incubation times with the same amount of purified RNase III shows an increase in the amount of dsRNA product in a similar size range of 12-15 basepairs. B. Increasing amounts of purified RNase III in μg levels leads to an increase in the amount of longer dsRNA product, as shown on a gel. C. Gel shows that decreasing amounts of RNase III in ng levels reduces amount of cleaved products.

FIG. 3A-B. Cleaved products from RNase III can induce RNA interference. A. Acrylamide gel shows the dsRNA products corresponding to La and LacZ generated after incubation with RNase III. The nucleic acid from the cut out region was eluted and then transfected into human cells. B. Graph showing the amount of fluorescence per cell of La expression observed after transfection of La-specific and La-nonspecific LacX RNase III products as compared to fluorescence in non-transfected negative controls (100).

FIG. 4A-B. Graph showing dose response of dsRNA product concentration (nM). Decreasing amounts of fluorescence were observed with increasing amounts of dsRNA product in both mouse 3T3 cells (FIG. 4A) and human HeLa cells (FIG. 4B). NT refers to a non-transfected control, which is a negative control (100). Increasing concentrations of dsRNA product show increased RNAi.

FIG. 5. 12-15 bp RNase III Digestion Products Elicit Silencing. A 200 bp GAPDH dsRNA (30 μg) was digested with RNase III (30 U) for 1 hour at RT. HeLa cells were transfected with 100 nM of the 12-15 bp RNase III generated GAPDH siRNAs or a 21 bp chemically synthesized GAPDH siRNA. GAPDH protein levels were monitored by immunofluorescence 48 hours after transfection and the resulting images were quantitated.

FIG. 6. RNase III siRNA Cocktails Show Specificity for Silencing. HeLa cells were transfected with 100 nM RNase III generated siRNAs to GAPDH. Immunofluorescence analysis of GAPDH, La, c-MYC, Cdk-2, Ku-90, and β-actin was performed 48 hours post transfection and subsequently quantitated.

### DESCRIPTION OF ILLUSTRATIVE EMBODIMENTS

The present invention is directed to compositions and methods relating to a labeled nucleic acid molecule that can be used in the process of RNA interference (RNAi). RNAi results in a reduction of expression of a particular target. Double stranded RNA has been shown to reduce gene expression of a target. A portion of one strand of the double stranded RNA is complementary to a region of the target's mRNA while another portion of the double stranded RNA molecule is identical to the same region of the target's mRNA. As discussed earlier, the RNA molecule of the invention is double stranded, which may be accomplished through two separate strands or a single strand having one region complementary to another region of the same strand. Discussed below are uses for the present invention—compositions, methods, and kits—and ways of implementing the invention.

### I. RNA Interference (RNAi)

RNA interference (also referred to as “RNA-mediated interference”)(RNAi) is a mechanism by which gene expression can be reduced or eliminated. Double stranded RNA (dsRNA) has been observed to mediate the reduction, which is a multi-step process. dsRNA activates post-transcriptional gene expression surveillance mechanisms that appear to function to defend cells from virus infection and transposon activity. (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001).

Interestingly, RNAi can be passed to progeny, both through injection into the gonad or by introduction into other parts of the body (including ingestion) followed by migration to the gonad. Several principles are worth noting (see Plasterk and Ketting, 2000). First, the dsRNA is typically directed to an exon, although some exceptions to this have been shown. Second, a homology threshold (probably about 80-85% over 200 bases) is required. Most tested sequences are 500 base pairs or greater, though sequences of 30 nucleotides or fewer evade the antiviral response in mammalian cells. (Baglioni et al., 1983; Williams, 1997). Third, the targeted mRNA is lost after RNAi. Fourth, the effect is non-stoichiometric, and thus incredibly potent. In fact, it has been estimated that only a few copies of dsRNA are required to knock down >95% of targeted gene expression in a cell (Fire et al., 1998).

Although the precise mechanism of RNAi is still unknown, the involvement of permanent gene modification or the disruption of transcription have been experimentally eliminated. It is now generally accepted that RNAi acts post-transcriptionally, targeting RNA transcripts for degradation. It appears that both nuclear and cytoplasmic RNA can be targeted. (Bosher et al., 2000).

Some of the uses for RNAi include identifying genes that are essential for a particular biological pathway, identifying disease-causing genes, studying structure function relationships, and implementing therapeutics and diagnostics. As with other types of gene inhibitory compounds, such as antisense and triplex forming oligonucleotides, tracking these potential drugs in vivo and in vitro is important for drug development, pharmacokinetics, biodistribution, macro and microimaging metabolism and for gaining a basic understanding of how these compounds behave and function. siRNAs have high specificity and may perhaps be used to knock out the expression of a single allele of a dominantly mutated diseased gene.

A. Polypeptides with RNAse III Domains

In certain embodiments, the present invention concerns compositions comprising at least one proteinaceous molecule, such as RNase III or a polypeptide having RNase III activity or an RNase III domain.

As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from 3 to 100 amino acids. All the “proteinaceous” terms described above may be used interchangeably herein.

In certain embodiments the size of the at least one proteinaceous molecule may comprise, but is not limited to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (can be found on the world wide web at ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

1. Functional Aspects

When the present application refers to the function or activity of RNase III, it is meant that the molecule in question has the ability to cleave a double-stranded RNA substrate into one or more dsRNA products.

2. Variants of RNase III and Proteins with RNase III Activity

Amino acid sequence variants of the polypeptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of an RNase III polypeptide or a protein having an RNase III domain, provided the biological activity of the protein is maintained.

The term “functionally equivalent codon” is used herein to refer to codons that encode the same amino acid, such as the six codons for arginine or serine, and also refers to codons that encode biologically equivalent amino acids (see Table 2, below).

TABLE 2
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 2 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of RNase III or a protein with an RNase III domain, but with altered and even improved characteristics.

3. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

4. Protein Purification

It may be desirable to purify RNase III, a protein with an RNase domain, or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques, including a Nickel column or using Histidine or glutathione tags. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

B. Nucleic Acids for RNAi

The present invention concerns double-stranded RNA capable of triggering RNAi. The RNA may be synthesized chemically or it may be produced recombinantly. They may be subsequently isolated and/or purified.

As used herein, the term “dsRNA” refers to a double-stranded RNA molecule. The molecule may be a single strand with intra-strand complementarity such that two portions of the strand hybridize with each other or the molecule may be two separate RNA strands that are partially or fully complementary to each other along one or more regions or along their entire lengths. Partially complementary means the regions are less than 100% complementary to each other, but that they are at least 50%, 60%, 70%, 80%, or 90% complementary to each other.

The siRNA provided by the present invention allows for the modulation and especially the attenuation of target gene expression when such a gene is present and liable to expression within a cell. Modulation of expression can be partial or complete inhibition of gene function, or even the up-regulation of other, secondary target genes or the enhancement of expression of such genes in response to the inhibition of the primary target gene. Attenuation of gene expression may include the partial or complete suppression or inhibition of gene function, transcript processing or translation of the transcript. In the context of RNA interference, modulation of gene expression is thought to proceed through a complex of proteins and RNA, specifically including small, dsRNA that may act as a “guide” RNA. The siRNA therefore is thought to be effective when its nucleotide sequence sufficiently corresponds to at least part of the nucleotide sequence of the target gene. Although the present invention is not limited by this mechanistic hypothesis, it is highly preferred that the sequence of nucleotides in the siRNA be substantially identical to at least a portion of the target gene sequence.

A target gene generally means a polynucleotide comprising a region that encodes a polypeptide, or a polynucleotide region that regulates replication, transcription or translation or other processes important tot expression of the polypeptide, or a polynucleotide comprising both a region that encodes a polypeptide and a region operably linked thereto that regulates expression. The targeted gene can be chromosomal (genomic) or extrachromosomal. It may be endogenous to the cell, or it may be a foreign gene (a transgene). The foreign gene can be integrated into the host genome, or it may be present on an extrachromosomal genetic construct such as a plasmid or a cosmid. The targeted gene can also be derived from a pathogen, such as a virus, bacterium, fungus or protozoan, which is capable of infecting an organism or cell. Target genes may be viral and pro-viral genes that do not elicit the interferon response, such as retroviral genes. The target gene may be a protein-coding gene or a non-protein coding gene, such as a gene which codes for ribosomal RNAs, splicosomal RNA, tRNAs, etc.

Any gene being expressed in a cell can be targeted. Preferably, a target gene is one involved in or associated with the progression of cellular activities important to disease or of particular interest as a research object. Thus, by way of example, the following are classes of possible target genes that may be used in the methods of the present invention to modulate or attenuate target gene expression: developmental genes (e.g. adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), oncogenes (e.g. ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TALI, TCL3 and YES), tumor suppresser genes (e.g. APC, BRCA1, BRCA2, MADH4, MCC, NF1, NF2, RB1, TP53 and WT1), and enzymes (e.g. ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases, DNA and RNA polymerases, galactosidases, glucanases, glucose oxidases, GTPases, helicases, hemicellulases, integrases, invertases, isomersases, kinases, lactases, lipases, lipoxygenases, lysozymes, pectinesterases, peroxidases, phosphatases, phospholipases, phosphorylases, polygalacturonases, proteinases and peptideases, pullanases, recombinases, reverse transcriptases, topoisomerases, xylanases).

The nucleotide sequence of the siRNA is defined by the nucleotide sequence of its target gene. The siRNA contains a nucleotide sequence that is essentially identical to at least a portion of the target gene. Preferably, the siRNA contains a nucleotide sequence that is completely identical to at least a portion of the target gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

A siRNA comprises a double stranded structure, the sequence of which is “substantially identical” to at least a portion of the target gene. “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides between such sequences. Identity can be readily calculated. See, for example: Computational Molecular Biology, Lesk, A. M., ed. Oxford University Press, New York, 1988; Biocomputing: Informatics and Genome Projects, Smith, D. W., ed., Academic Press, New York, 1993; and the methods disclosed in WO 99/32619, WO 01/68836, WO 00/44914, and WO 01/36646, specifically incorporated herein by reference. While a number of methods exist for measuring identity between two nucleotide sequences, the term is well known in the art. Methods for determining identity are typically designed to produce the greatest degree of matching of nucleotide sequence and are also typically embodied in computer programs. Such programs are readily available to those in the relevant art. For example, the GCG program package (Devereux et al.), BLASTP, BLASTN, and FASTA (Atschul et al.) and CLUSTAL (Higgins et al., 1992; Thompson, et al., 1994).

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. It is preferred that there be 100% sequence identity between the dsRNA for use as siRNA and at least 15 contiguous nucleotides of the target gene, although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

RNA (ribonucleic acid) is known to be the transcription product of a molecule of DNA (deoxyribonucleic acid) synthesized under the action of an enzyme, DNA-dependent RNA polymerase. There are diverse applications of the obtaining of specific RNA sequences, such as, for example, the synthesis of RNA probes or of oligoribonucleotides (Milligan et al.), or the expression of genes (see, in particular, Steen et al., Fuerst, et al. and Patent Applications WO 91/05,866 and EP 0,178,863), or alternatively gene amplification as described by Kievits, et al. and Kwoh et al. or in Patent Applications WO 88/10,315 and WO 91/02,818, and U.S. Pat. No. 5,795,715, all of which are expressly incorporated herein by reference.

One of the distinctive features of most DNA-dependent RNA polymerases is that of initiating RNA synthesis according to a DNA template from a particular start site as a result of the recognition of a nucleic acid sequence, termed a promoter, which makes it possible to define the precise localization and the strand on which initiation is to be effected. Contrary to DNA-dependent DNA polymerases, polymerization by DNA-dependent RNA polymerases is not initiated from a 3′-OH end, and their natural substrate is an intact DNA double strand.

Compared to bacterial, eukaryotic or mitochondrial RNA polymerases, phage RNA polymerases are very simple enzymes. Among these, the best known are the RNA polymerases of bacteriophages T7, T3 and SP6. These enzymes are very similar to one another, and are composed of a single subunit of 98 to 100 kDa. Two other phage polymerases share these similarities: that of Klebsiella phage K11 and that of phage BA14 (Diaz et al.). Any DNA dependent RNA polymerase is expected to perform in conjunction with a functionally active promoter as desired in the present invention. These include, but are not limited to the above listed polymerases, active mutants thereof, E. coli RNA polymerase, and RNA polymerases I., II, and III from a variety of eukaryotic organisms.

Initiation of transcription with T7, SP6 RNA and T3 RNA Polymerases is highly specific for the T7, SP6 and T3 phage promoters, respectively. The properties and utility of these polymerases are well known to the art. Their properties and sources are described in U.S. Pat. Nos. (T7) 5,869,320; 4,952,496; 5,591,601; 6,114,152; (SP6) 5,026,645; (T3) 5,102,802; 5,891,681; 5,824,528; 5,037,745, all of which are expressly incorporated herein by reference.

Reaction conditions for use of these RNA polymerases are well known in the art, and are exemplified by those conditions provided in the examples and references. The result of contacting the appropriate template with an appropriate polymerase is the synthesis of an RNA product, which is typically single-stranded. Although under appropriate conditions, double stranded RNA may be made from a double stranded DNA template. See U.S. Pat. No. 5,795,715, incorporated herein by reference. The process of sequence specific synthesis may also be known as transcription, and the product the transcript, whether the product represents an entire, functional gene product or not.

dsRNA for use as siRNA may also be enzymatically synthesized through the use of RNA dependent RNA polymerases such as Q beta replicase, Tobacco mosaic virus replicase, brome mosaic virus replicase, potato virus replicase, etc. Reaction conditions for use of these RNA polymerases are well known in the art, and are exemplified by those conditions provided in the examples and references. Also see U.S. Pat. No. RE35,443, and U.S. Pat. No. 4,786,600, both of which are incorporated herein by reference. The result of contacting the appropriate template with an appropriate polymerase is the synthesis of an RNA product, which is typically double-stranded. Employing these RNA dependent RNA polymerases therefore may utilize a single stranded RNA or single stranded DNA template. If utilizing a single stranded DNA template, the enzymatic synthesis results in a hybrid RNA/DNA duplex that is also contemplated as useful as siRNA.

The templates for enzymatic synthesis of siRNA are nucleic acids, typically, though not exclusively DNA. A nucleic acid may be made by any technique known to one of ordinary skill in the art. Non-limiting examples of synthetic nucleic acid, particularly a synthetic oligonucleotide, include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. Pat. No. 5,705,629, each incorporated herein by reference. A non-limiting example of enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells (see for example, Sambrook, 2001, incorporated herein by reference).

The term “nucleic acid” will generally refer to at least one molecule or strand of DNA, RNA or a derivative or mimic thereof, comprising at least one nucleotide base, such as, for example, a naturally occurring purine or pyrimidine base found in DNA (e.g., adenine “A,” guanine “G,” thymine “T,” and cytosine “C”) or RNA (e.g. A, G, uracil “U,” and C). The term “nucleic acid” encompasses the terms “oligonucleotide” and “polynucleotide.” These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

As will be appreciated by one of skill in the art, the useful form of nucleotide or modified nucleotide to be incorporated will be dictated largely by the nature of the synthesis to be performed. Thus, for example, enzymatic synthesis typically utilizes the free form of nucleotides and nucleotide analogs, typically represented as nucleotide triphospates, or NTPs. These forms thus include, but are not limited to aminoallyl UTP, pseudo-UTP, 5-I-UTP, 5-I-CTP, 5-Br-UTP, alpha-S ATP, alpha-S CTP, alpha-S GTP, alpha-S UTP, 4-thio UTP, 2-thio-CTP, 2′NH₂ UTP, 2′NH₂ CTP, and 2′ F UTP. As will also be appreciated by one of skill in the art, the useful form of nucleotide for chemical syntheses may be typically represented as aminoallyl uridine, pseudo-uridine, 5-I-uridine, 5-I-cytidine, 5-Br-uridine, alpha-S adenosine, alpha-S cytidine, alpha-S guanosine, alpha-S uridine, 4-thio uridine, 2-thio-cytidine, 2′NH₂ uridine, 2′NH₂ cytidine, and 2′ F uridine. In the present invention, the listing of either form is non-limiting in that the choice of nucleotide form will be dictated by the nature of the synthesis to be performed. In the present invention, then, the inventors use the terms aminoallyl uridine, pseudo-uridine, 5-I-uridine, 5-I-cytidine, 5-Br-uridine, alpha-S adenosine, alpha-S cytidine, alpha-S guanosine, alpha-S uridine, 4-thio uridine, 2-thio-cytidine, 2′NH₂ uridine, 2′NH₂ cytidine, and 2′ F uridine generically to refer to the appropriate nucleotide or modified nucleotide, including the free phosphate (NTP) forms as well as all other useful forms of the nucleotides.

In certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises a nucleic acid, and/or encodes a polypeptide or peptide-coding sequences of a gene that is defective or mutated in a hematopoietic and lympho-hematopoietic disorder. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. Thus, a “truncated gene” refers to a nucleic acid sequence that is missing a stretch of contiguous nucleic acid residues.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and/or so on. For a 15-mer, the nucleic acid segments correspond to bases 1 to 15, 2 to 16, 3 to 17 . . . and/or so on. For a 20-mer, the nucleic segments correspond to bases 1 to 20, 2 to 21, 3 to 22 . . . and/or so on.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended protocol.

To obtain the RNA corresponding to a given template sequence through the action of an RNA polymerase, it is necessary to place the target sequence under the control of the promoter recognized by the RNA polymerase.

The spacing between promoter elements frequently is flexible, so that promoter function is preserved when elements are inverted or moved relative to one another. The spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

T7, T3, or SP6 RNA polymerases display a high fidelity to their respective promoters. The natural promoters specific for the RNA polymerases of phages T7, T3 and SP6 are well known. Furthermore, consensus sequences of promoters are known to be functional as promoters for these polymerases. The bacteriophage promoters for T7, T3, and SP6 consist of 23 bp numbered −17 to +6, where +1 indicates the first base of the coded transcript. An important observation is that, of the +1 through +6 bases, only the base composition of +1 and +2 are critical and must be a G and purine, respectively, to yield an efficient transcription template. In addition, synthetic oligonucleotide templates only need to be double-stranded in the −17 to −1 region of the promoter, and the coding region can be all single-stranded. (See Milligan et al.) This can reduce the cost of synthetic templates, since the coding region (i.e., from +1 on) can be left single-stranded and the short oligonucleotides required to render the promoter region double-stranded can be used with multiple templates. A further discussion of consensus promoters and a source of naturally occurring bacteriophage promoters is U.S. Pat. No. 5,891,681, specifically incorporated herein by reference.

Use of a T7, T3 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

When made in vitro, siRNA is formed from one or more strands of polymerized ribonucleotide. When formed of only one strand, it takes the form of a self-complementary hairpin-type or stem and loop structure that doubles back on itself to form a partial duplex. The self-duplexed portion of the RNA molecule may be referred to as the “stem” and the remaining, connecting single stranded portion referred to as the “loop” of the stem and loop structure. When made of two strands, they are substantially complementary.

It is contemplated that the region of complementarity in either case is at least 5 contiguous residues, though it is specifically contemplated that the region is at least or at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides. It is further understood that the length of complementarity between the dsRNA and the targeted mRNA may be any of the lengths identified above. Included within the term “dsRNA” is small interfering RNA (siRNA), which are generally 12-15 or 21-23 nucleotides in length and which possess the ability to mediate RNA interference. It is contemplated that RNase III dsRNA products of the invention may be 12, 13, 14, 15, 16, 17, 18, 19, 20, 21, 22, 23, 24, 25, 26, 27, 28, 29, 30 or more basepairs in length.

dsRNA capable of triggering RNAi has one region that is complementary to the targeted mRNA sequence and another region that is identical to the targeted mRNA sequence. Of course, it is understood that an mRNA is derived from genomic sequences or a gene. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants.

A dsRNA may be of the following lengths, or be at least or at most of the following lengths: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It will be understood that these lengths refer either to a single strand of a two-stranded dsRNA molecule or to a single stranded dsRNA molecule having portions that form a double-stranded molecule.

Furthermore, outside regions of complementarity, there may be a non-complementarity region that is not complementary to another region in the other strand or elsewhere on a single strand. Non-complementarity regions may be at the 3′, 5′ or both ends of a complementarity region and they may number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 5, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more bases.

The term “recombinant” may be used and this generally refers to a molecule that has been manipulated in vitro or that is the replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The use of “dsRNA” encompasses both “oligonucleotides” and “polynucleotides,” unless otherwise specified.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

1. Nucleic Acid Molecules

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. In the table below, non-limiting, purine and pyrimidine derivatives and analogs are also provided.

TABLE 3
Purine and Pyrmidine Derivatives or Analogs

| Abbr. | Modified base description |
|---|---|
| ac4c | 4-acetylcytidine |
| Chm5u | 5-(carboxyhydroxylmethyl) uridine |
| Cm | 2′-O-methylcytidine |
| Cmnm5s2u | 5-carboxymethylamino-methyl-2-thiouridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-ribofuranosyl-2- |
| | methylthiopurine-6-yl)carbamoyl)threonine |
| Mt6a | N-((9-beta-ribofuranosylpurine-6-yl)N- |
| | methyl-carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

b. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

c. Nucleotides

As used herein, a “nucleotide” refers to a nucleoside further comprising a “backbone moiety.” A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. Other types of attachments are known in the art, particularly when a nucleotide comprises derivatives or analogs of a naturally occurring 5-carbon sugar or phosphorus moiety.

d. Nucleic Acid Analogs

A nucleic acid may comprise, or be composed entirely of, a derivative or analog of a nucleobase, a nucleobase linker moiety and/or backbone moiety that may be present in a naturally occurring nucleic acid. dsRNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moeity which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugaged to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and 5,480,980 (7-deaza-2′ deoxyguanosine nucleotides and nucleic acid analogs thereof).

2. Preparation of Nucleic Acids

The present invention concerns various nucleic acids in different embodiments of the invention. There are a variety of ways to generate a dsRNA that can be a substrate for a polypeptide with RNase III activity. In some embodiments, dsRNA is created by transcribing a DNA template. The DNA template may be comprised in a vector or it may be a non-vector template. Alternatively, a dsRNA may be created by hybridizing two synthetic, complementary RNA molecules or hybridizing a single synthetic RNA molecule with at least one complementarity region. Such nucleic acids may be made by any technique known to one of ordinary skill in the art, such as for example, chemical synthesis, enzymatic production or biological production.

a. Vectors

Nucleic acids of the invention, particularly DNA templates, may be produced recombinantly. Protein and polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (2001) and Ausubel et al., 1994, both incorporated by reference. A vector may encode non-template sequences such as a tag or label. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter (examples include the bacterial promoters SP6, T3, and T7), which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Other elements of a vector are well known to those of skill in the art. A vector may include a polyadenylation signal, an initiation signal, an internal ribosomal binding site, a multiple cloning site, a selective or screening marker, a termination signal, a splice site, an origin of replication, or a combination thereof.

b. In Vitro Synthesis of dsRNA

A DNA template may be used to generate complementing RNA molecule(s) to generate a double-stranded RNA molecule that can be a substrate for RNase III. One or two DNA templates may be employed to generate a dsRNA. In some embodiments, the DNA template can be part of a vector or plasmid, as described herein. Alternatively, the DNA template for RNA may be created by an amplification method.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred. Pairs of primers designed to selectively hybridize to nucleic acids corresponding to the target gene are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification are conducted until a sufficient amount of product is produced.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety. A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety. Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase copies the replicative sequence which may then be detected. An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). EP Application 329 822 disclose a nucleic acid amplification process involving cyclically synthesizing ssRNA, ssDNA, and dsDNA, which may be used in accordance with the present invention. PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

c. Chemical Synthesis

Nucleic acid synthesis is performed according to standard methods. See, for example, Itakura and Riggs (1980). Additionally, U.S. Pat. No. 4,704,362, U.S. Pat. No. 5,221,619, and U.S. Pat. No. 5,583,013 each describe various methods of preparing synthetic nucleic acids. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligonucleotides (Gillam et al., 1978; Gillam et al., 1979). Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers.

Phosphoramidite chemistry (Beaucage and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

3. Nucleic Acid Purification

A nucleic acid may be purified on polyacrylamide gels, cesium chloride centrifugation gradients, or by any other means known to one of ordinary skill in the art (see for example, Sambrook (2001), incorporated herein by reference). Alternatively, a column, filter, or cartridge containing an agent that binds to the nucleic acid, such as a glass fiber, may be employed.

Following any amplification or transcription reaction, it may be desirable to separate the amplification or transcription product from the template and/or the excess primer. In one embodiment, products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Nucleic Acid Transfer

Suitable methods for nucleic acid delivery to effect RNAi according to the present invention are believed to include virtually any method by which a nucleic acid (e.g., DNA, RNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

There are a number of ways in which expression vectors may be introduced into cells to generate dsRNA. In certain embodiments of the invention, the expression vector comprises a virus or engineered vector derived from a viral genome, while in other embodiments, it is a nonviral vector. Other expression systems are also readily available.

5. Host Cells and Target Cells

The cell containing the target gene may be derived from or contained in any organism (e.g., plant, animal, protozoan, virus, bacterium, or fungus). The plant may be a monocot, dicot or gynmosperm; the animal may be a vertebrate or invertebrate. Preferred microbes are those used in agriculture or by industry, and those that a pathogenic for plants or animals. Fungi include organisms in both the mold and yeast morphologies. Examples of vertebrates include fish and mammals, including cattle, goat, pig, sheep, hamster, mouse, rate and human; invertebrate animals include nematodes, insects, arachnids, and other arthropods. Preferably, the cell is a vertebrate cell. More preferably, the cell is a mammalian cell.

The cell having the target gene may be from the germ line or somatic, totipotent or pluripotent, dividing or non-dividing, parenchyma or epithelium, immortalized or transformed, or the like. The cell can be a gamete or an embryo; if an embryo, it can be a single cell embryo or a constituent cell or cells from a multicellular embryo. The term “embryo” thus encompasses fetal tissue. The cell having the target gene may be an undifferentiated cell, such as a stem cell, or a differentiated cell, such as from a cell of an organ or tissue, including fetal tissue, or any other cell present in an organism. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells, of the endocrine or exocrine glands.

As used herein, the terms “cell,” “cell line,” and “cell culture” may be used interchangeably. All of these terms also include their progeny, which is any and all subsequent generations formed by cell division. It is understood that all progeny may not be identical due to deliberate or inadvertent mutations. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a small, interfering RNA or a template construct encoding such an RNA has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed or contacted with a nucleic acid delivery composition and/or an additional agent. The tissue may be part or separated from an organism. In certain embodiments, a tissue and its constituent cells may comprise, but is not limited to, blood (e.g., hematopoietic cells (such as human hematopoietic progenitor cells, human hematopoietic stem cells, CD34⁺ cells CD4⁺ cells), lymphocytes and other blood lineage cells), bone marrow, brain, stem cells, blood vessel, liver, lung, bone, breast, cartilage, cervix, colon, cornea, embryonic, endometrium, endothelial, epithelial, esophagus, facia, fibroblast, follicular, ganglion cells, glial cells, goblet cells, kidney, lymph node, muscle, neuron, ovaries, pancreas, peripheral blood, prostate, skin, skin, small intestine, spleen, stomach, testes.

In certain embodiments, the host cell or tissue may be comprised in at least one organism. In certain embodiments, the organism may be, human, primate or murine. In other embodiments the organism may be any eukaryote or even a prokayrote (e.g., a eubacteria, an archaea), as would be understood by one of ordinary skill in the art (see, for example, webpage http://phylogeny.arizona.edu/tree/phylogeny.html). One of skill in the art would further understand the conditions under which to incubate all of the above described host cells to maintain them and to permit their division to form progeny.

6. Labels and Tags

dsRNA or resulting siRNA may be labeled with a radioactive, enzymatic, colorimetric, or other label or tag for detection or isolation purposes. Nucleic acids may be labeled with fluorescence in some embodiments of the invention. The fluorescent labels contemplated for use as conjugates include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red.

It is contemplated that dsRNA may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

A number of techniques for visualizing or detecting labeled dsRNA are readily available. The reference by Stanley T. Crooke, 2000 has a discussion of such techniques (Chapter 6) which is incorporated by reference. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997, spectroscopy, capillary gel electrophoresis (Cummins et al., 1996), spectroscopy; mass spectroscopy; radiological techniques; and mass balance techniques. Alternatively, nucleic acids may be labeled or tagged to allow for their efficient isolation. In other embodiments of the invention, nucleic acids are biotinylated.

7. Libraries and Arrays

The present methods and kits may be employed for high volume screening. A library of either dsRNA or candidate siRNA can be created using methods of the invention. This library may then be used in high throughput assays, including microarrays. Specifically contemplated by the present inventors are chip-based nucleic acid technologies such as those described by Hacia et al. (1996) and Shoemaker et al. (1996). Briefly, these techniques involve quantitative methods for analyzing large numbers of genes rapidly and accurately. By using fixed probe arrays, one can employ chip technology to segregate target molecules as high density arrays and screen these molecules on the basis of hybridization (see also, Pease et al., 1994; and Fodor et al, 1991). The term “array” as used herein refers to a systematic arrangement of nucleic acid. For example, a nucleic acid population that is representative of a desired source (e.g., human adult brain) is divided up into the minimum number of pools in which a desired screening procedure can be utilized to detect or deplete a target gene and which can be distributed into a single multi-well plate. Arrays may be of an aqueous suspension of a nucleic acid population obtainable from a desired mRNA source, comprising: a multi-well plate containing a plurality of individual wells, each individual well containing an aqueous suspension of a different content of a nucleic acid population. Examples of arrays, their uses, and implementation of them can be found in U.S. Pat. Nos. 6,329,209, 6,329,140, 6,324,479, 6,322,971, 6,316,193, 6,309,823, 5,412,087, 5,445,934, and 5,744,305, which are herein incorporated by reference.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995a. See also DeRisi et al., 1996; Shalon et al., 1996; Schena et al., 1995b. Other methods for making microarrays, e.g., by masking (Maskos et al., 1992), may also be used. In principal, any type of array, for example, dot blots on a nylon hybridization membrane (see Sambrook et al., 1989, which is incorporated in its entirety for all purposes), could be used, although, as will be recognized by those of skill in the art, very small arrays will be preferred because hybridization volumes will be smaller.

### III. Kits

Any of the compositions described herein may be comprised in a kit. In a non-limiting example, reagents for generating siRNA molecules are included in a kit. The kit may further include reagents for creating or synthesizing the dsRNA. The kits will thus comprise, in suitable container means, a polypeptide with RNase III activity for generating siRNA. It may also include one or more buffers, such as a nuclease buffer, transcription buffer, or a hybridization buffer, compounds for preparing the DNA template or the dsRNA, and components for isolating the resultant template, dsRNA, or siRNA. Other kits of the invention may include components for making a nucleic acid array comprising siRNA, and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Such kits may also include components that facilitate isolation of the DNA template, long dsRNA, or siRNA. It may also include components that preserve or maintain the nucleic acids or that protect against their degradation. Such components may be RNAse-free or protect against RNAses, such as RNase inhibitors. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following in addition to a polypeptide with RNase III activity: 1) RNase III buffer; 2) Control dsRNA, including but not limited to, GAPDH siRNA or c-myc siRNA (shown in Examples); 3) SP6, T3, and/or T7 polymerase; 4) SP6, T3, and/or T7 polymerase buffer; 5) dNTPS and/or NTPs; 6) nuclease-free water; 7) RNase-free containers, such as 1.5 ml tubes; 8) RNase-free elution tubes; 9) glycogen; 10) ethanol; 11) sodium acetate; 12) ammonium acetate; 13) agarose or acrylamide gel; 14) nucleic acid size marker; 15) RNase-free tube tips; or 16) RNase or DNase inhibitors.

It is contemplated that such reagents are embodiments of kits of the invention. Such kits, however, are not limited to the particular items identified above and may include any labeling reagent or reagent that promotes or facilitates the labeling of a nucleic acid to trigger RNAi.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Bacterial RNase III Cleaves Long dsRNA into Small Fragments

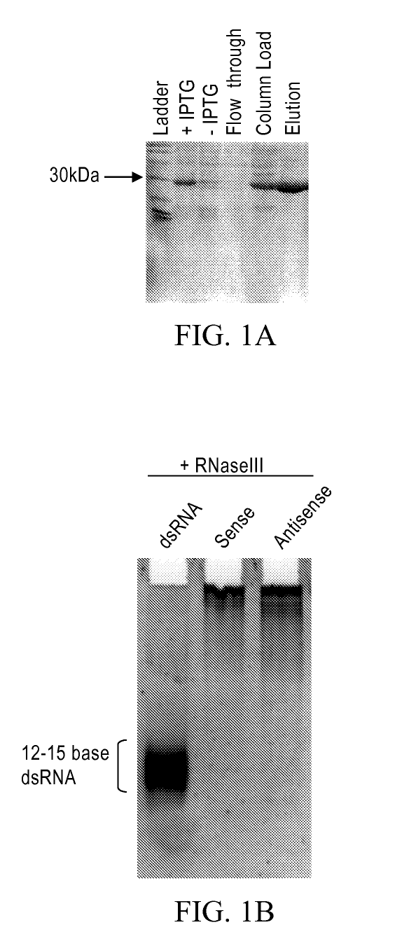

Bacterial RNase III cleaves long dsRNA into RNAs that are 12-15 bp in length. The His-tagged bacterial RNase III was purified as follows: (From a 1-liter culture we made 13 mg of total RNase III protein with 10 mls of a 1.3 mg/ml solution). First, dilution streak the RNase III strain of bacteria BL21 (DE3) E. coli containing the pET-11a with the mc gene cloned into Nde I and Bam HI sites onto an agar plate containing LB-amp (50-100 μg/ml) and grow at 37° C. overnight. This plasmid contains the rnc gene (i.e., RNaseIII gene) under the control of an IPTG inducible T7 promoter and translation initiation signal. From a single colony, inoculate 20 ml of LB and grow at 37° C. overnight with vigorous aeration. Inoculate 1 liter of LB-amp with 20 ml of the overnight culture from step 2. Let this culture grow until it reaches an OD of 0.3-0.4 at OD 600 nm. Induce cells with IPTG (final concentration of 0.5-1 mM) and let grow for 4 hours. Harvest cells by centrifugation and store at −80° C. or proceed to protein purification. Suspend the cell pellet in 30 mls of buffer A (500 mM NaCl, 20 mM Tris-HCl (pH 8.0) and 5 mM imidazole). Sonicate on Ice until lysate clarifies. Centrifuge at 7000 rpm for 20 minutes in an SS34 rotor. Apply protein solution to Ni-NTA column that has been washed and equilibrated with buffer A. Wash column with 10 column volumes of buffer A. Wash column with 6 column volumes of buffer A containing 60 mM Imidazole. Elute with 150 ml of elution buffer (1 M NaCl, 20 mM Tris-HCL (pH 8.0) and 400 mM Imidazole). Collected 1 ml fractions and combine those with the highest protein concentration. Dialyze against buffer D1 (1 M NaCl, 60 mM Tris-HCL (pH 8.0), and 400 mM Imidazole) for 2 hours. Dialyze against buffer D2 for (1 M NaCl and 60 mM Tris-HCl (pH8.0) 2 hours. Dialyze against buffer D3 (1 M NaCl 60 mM Tris-HCl (pH 8.0), 1 mM EDTA, and 1 mM DTT for 12-16 hours. Add glycerol to bring its concentration to 50%. Purified RNase III was run on a 15% acrylamide gel containing SDS along side a ladder with a marked 30 Kda size range, cell lysate prior to IPTG induction, after 4 hours of IPTG induction, flow through of the Ni-NTA column, column load and elution (FIG. 1A). The elution shown is a combination of peaks that came off the column that had the highest protein concentration as determined by OD. The purified RNase III was dialyzed, diluted in glycerol and used for RNase III digestion reactions. The RNA that was used for RNase III cleavage is derived from our pdp c-fos vector that was transcribed with T7 and T3 to produce a 250-base RNA that corresponds to the following sequence of the c-fos gene:

(SEQ ID NO: 2)
tacgatttaggtgacactatagaatacacggaattaatacgactcacta
tagggaattaccctcactaaagggaggaagctgcaattgggatgcaagc
tttccacatctggcacagagcgggaggtctctgagccactgggcctaga
tgatgccggaaacaagaagtcatcaaagggttctgccttcagctccacg
ttgctgatgctcttgactggctccaaggatggcttgggctcagggtcgt
tgagaaggggcagggtgaaggcctcctcagactctggggtggaagcctc
aggcagacctccagtcaaatccagggaggccacagacatctcctctggg
aagccaagaatt.

The sense and antisense strands were hybridized by incubating equal molar amounts of the sense and antisense strands in 100 mM NaCl, 20 mM Tris pH 7.0 and 1 mM EDTA heating to 95° C. for 10 minutes in a heat block and let cool to room temperature slowly. The double strand c-fos RNA, or sense and antisense strands of the c-fos RNA were incubated with recombinant RNase III at 37° C. for 1 hour in 30 mM Tris pH 8.0, 160 mM NaCl, 0.1 mM EDTA, 0.1 mM DTT, and 5 mM MgCl₂. The samples were phenol chloroform extracted, ethanol precipitated, loaded and run on a 15% non-denaturing acrylamide gel. The gel was stained with ethidium bromide and analyzed using an alphaimager 2200 gel documentation system (FIG. 1B). The long dsRNA substrate was cleaved into 12-15 basepair fragments.

### Example 2

### Limited RNase III Digestion Varies Size of Product

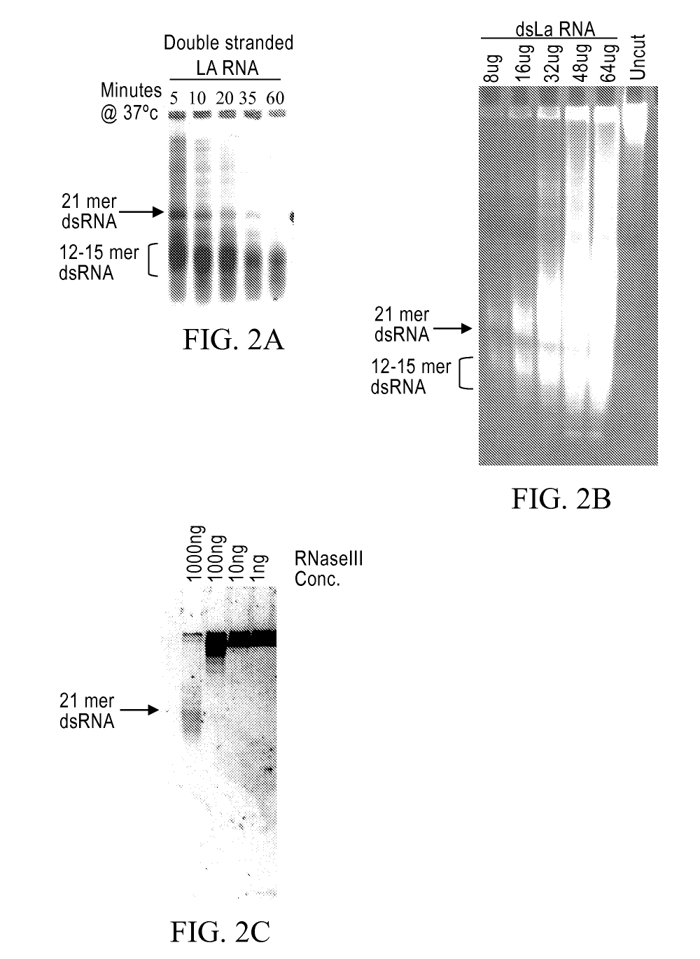

Limited RNase III digestion leads to dsRNA with sizes that range from 12-30 bases in length with a band in the 21 base region. FIG. 2A. A 200-base dsRNA that corresponds to the human La mRNA was produced as follows. PCR from a HeLa cell cDNA was performed using 4 μl dNTP's (2.5 mM dATP, dGTP, dCTP, dTTP), 4 μl, Taq 0.5 μl, 0.5 ml primers 5′-AAT TTA ATA CGA CTC ACT ATA GGA AGC ATT GAG CAA ATC C-3′ (SEQ ID NO:3) and 5′-AAT TTA ATA CGA CTC ACT ATA GGC TTC TGG CCA GGG GTC TC (SEQ ID NO:4) (both primers at 100 pmole/μl), 38.5 μl water, 10×PCR buffer (100 mM Tris pH 8.3, 500 mM KCl, and 15 mM MgCl₂). The PRC reaction was cycled 35 times at 95° C. for 30 seconds, 48° C. for 30 seconds and at 72° C. for 30 seconds. Then one cycle for 10 minutes at 72° C. all in a MJ Research minicycler. The 200 base PCR product was gel purified using Qiagen minielute gel elution kit (cat #28604). The gel purified PCR products were then phenol chloroform extracted, ethanol precipitated and suspended into nuclease free water. The PCR products containing T7 promoters were then used for in vitro transcription. Transcription using the MegaScript® kit from Ambion (Cat # 1334). 9 μg of La dsRNA was incubated with 2 μl of 5× reaction buffer (100 mM Tris, pH 7.5, 25 mM MgCl₂ and 1 mM CaCl₂), 12 μl of nuclease free water and 1.3 mg of RNase III at 37° C. for the indicated times. Arrows indicate region of the gel that represents a 21 base siRNA and siRNA extending 12-15 bases in length. (FIG. 2B). The 200 base double stranded RNA corresponding to the human Lac Z mRNA was produced as follows: dNTP mix (2.5 mM dATP, dGTP, dCTP, dTTP), 4 μl, Taq 0.5 μl, 0.5 μl primers 5′-AAT TTA ATA CGA CTC ACT ATA GGT ACC AGA AGC GGT GCC GG (SEQ ID NO:5) and 5′-AAT TTA ATA CGA CTC ACT ATA GGC AAA CGA CTGTCC TGG CCG T (SEQ ID NO:6) (100 pmole/μl), 38.5 μl water, 10×PCR buffer (100 mM Tris pH 8.3, 500 mM KCl, and 15 mM MgCl₂). The PRC reaction was cycled 35 times at 95° C. for 30 seconds, 48° C. for 30 seconds and at 72° C. for 30 seconds. Then one cycle for 10 minutes at 72° C. all in a MJ Research minicycler. The 200-base PCR product was gel purified using Qiagen minielute gel elution kit (cat #28604). The gel purified PCR products were then phenol chloroform extracted, ethanol precipitated and suspended into nuclease free water. The LacZ 200 base PCR product containing T7 promoters were then used for in vitro transcription. Transcription using the MegaScript® kit from Ambion (Cat # 1334). Following transcription, the LacZ double stranded RNA was cleaved with RNAse III as follows: Increasing amounts of the 200 base pair double stranded Lac Z RNA was incubated with 1 μg of RNase III at 37° C. for 1 hour. An undigested amount of the 200 base double stranded La RNA was used as a control (FIG. 2C). La dsRNA was digested with differing amounts of RNase III listed in the figure represented on the gel is the size that a 21 base siRNA migrates. These data indicate that RNase III digested product size can be manipulated by using different amount of enzyme, substrate and reaction time. We also propose manipulating reaction buffers to find those condition that RNase III gives the most amount of 21 base fragments in. These experiment demonstrate that reaction conditions can be manipulated so that more or less 21 base pair fragments are the bulk of the product generated by the RNase III enzyme. This may be important in generating more defined length siRNA.

### Example 3

### RNase III Products can Induce Gene Silencing in Mammalian Cells

PCR of LA and Lac Z was performed according to the procedure described in Example 2. Following transcription, the Lac Z and La RNA was cleaved with RNAse III as follows: 6.5 μg of double stranded RNA, 1 μl of RNase III, 10 μl of 5× RNase III buffer (150 mM Tris, pH 8.0, 800 mM NaCl, 0.5 mM EDTA, 0.5 mM DTT, and 50 mM MgCl₂), and 34 μl Nuclease free water were mixed and incubated at 37° C. for 4 hours. Following the reaction the RNA was phenol-chloroform extracted, ethanol precipitated and run on a 15% acrylamide gel. The gel slice containing the RNase III cleavage products ranging in size between 15-21 bases was cut out of the gel and incubated overnight with rotation at 37° C. in 50 mM Tris, pH 7.6, 0.1% SDS and 400 mM NaCl. Following overnight incubation, the RNA was precipitated with ethanol, dried and suspended in nuclease free water.

Gel purified siRNA was then used for transfection into HeLa cells. Transfection were performed as follows: Hela-S3 cells were plated in triplicate and transfected with either La or Lac Z as follows: Hela-S3 cells were plated at 50,000 cells per well into a 24 well tissue culture plate containing a 12 mm-glass cover slip, in triplicate. The cells are transfected 24 hours after plating using Oligofectamine transfection reagent (Invitrogen cat #12252-011) as follows: First, 40 ul Opti-MEMO is added to a sterile round bottom polystyrene 12×75 mm tube. Next, RNAse III digested RNA was added to produce a final concentration of 100 nM in 250 μl final transfection volume. In a separate tube, 6.0 μl of Opti-MEM® is added to a sterile round bottom polystyrene 12×75 mm tube. Next, 1.5 μl of Oligofectamine reagent was added and the mix was incubated at room temperature for 10 minutes. The two tubes were then mixed and incubate at room temperature for 20 minutes. During incubation, growth media was removed from cells and 200 μl of Opti-MEM® was added. T transfection mixture was added to the 200 Opti-MEM® on the cells and incubated in a tissue culture incubator at 37° C. with 5% CO₂ for 4 hours. After four hours, 1.0 ml of HeLa growth media was added (DMEM, 10% FBS, 10% Penicillin-Streptomycin). The cells were harvested at 48 hours following transfection and immunofluorescence for La was performed.

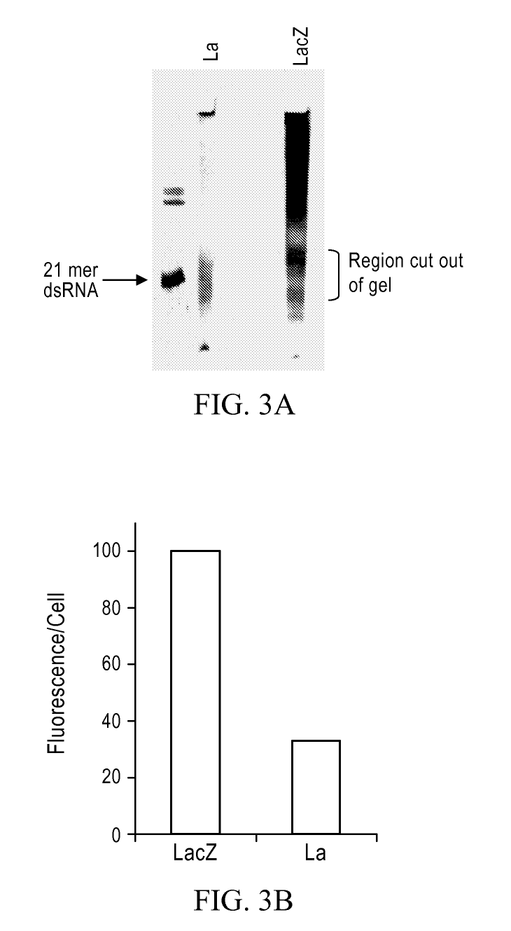

For immunofluorescence, the growth media was removed, the cells were washed with 1 ml of 1×PBS, and 400 μl of fresh 4% Paraformaldehyde/PBS (Paraformaldehyde, Sigma Cat #P-6148) was added into each well and incubated for 5 minutes at room temperature. After the paraformaldehyde incubation, the cells were washed with 1 ml of 1×PBS, and permiabilized by adding 500 μl of in 0.1% Triton X-100/PBS (Triton X-100, Sigma Cat # T-9284) for 5 minutes. The cells were then washed with 1 ml of 1×PBS, and 500 μl of 3% BSA/PBS (BSA, Sigma Cat # B-4287) was added and incubate for 1 hour at room temperature. The cells were then washed with 1 ml of 1×PBS, 500 μl of primary antibody (Transduction Labs cat #L69320) diluted in 1×PBS (1:500) and incubate for 1 hour at room temperature on Nutator. The primary antibody was removed and the cells were washed with 1 ml of 1×PBS. The secondary antibody (Jackson ImmunoResearch, Fluorescein (FITC)-conjugated affinity pure donkey anti-mouse IgG, Cat # 715-095-150) was then added and incubate for 1 hour at room temperature. The cells were then washed with 1 ml of 1×PBS, washed with 300 μl of nuclease free dH₂O and mounted onto glass slides using VectaShield with DAPI (Vector Labs, cat# H-1200). Fluorescent signal was detected using an Olympus BX60 microscope and quantified using MetaMorph software. The RNase III cleaved La product demonstrated La-specific reduction in gene expression. (FIG. 3).

### Example 4

### Dose Response for the Gene Silencing of La Using RNasIII Cleavage Products

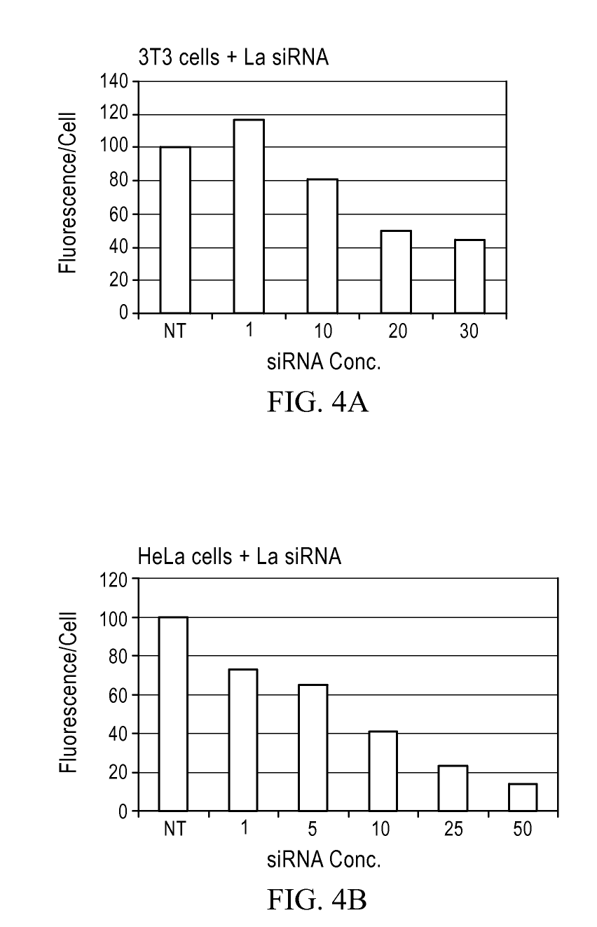

The La 200-base double stranded double stranded RNA was generated as described in Example 2. However, instead of gel purification, following the siRNA cleavage, RNase III products were run over a size exclusion column microcon 100 (Millipore cat #42412) that separates the short siRNA from the long undigested double stranded product. Following column purification, the siRNA was phenol chloroform extracted, ethanol precipitated, suspended in water and the nucleic acid concentration was determined. Different concentrations of RNase III digested product were transfected into NIH3T3 (FIG. 4A) or HeLa cells (FIG. 4B) using oligofectamine (Invitrogen cat# 12252-011) at the indicated concentrations that represent the final siRNA concentration in the tissue culture media.

The cells were transfected and analyzed as described in Example 3 using immunofluorescence and MetaMorph. These data demonstrate that siRNA produced using RNase III causes a clear dose response and is effective at low concentrations. The concentration of the individual siRNA in the population is at least 10-fold lower than what is labeled on the graph because siRNA generated from a 200-base double stranded RNA is mixture of approximately 10-15 different siRNA molecules. Thus each individual siRNA in this population is 10-15 fold lower than what is described for the total siRNA concentration and suggests that the siRNA generated using RNase III may be more potent than any one single siRNA transfected alone. Thus, at an individual siRNA concentration of 1 nM, a 41% decrease in La protein expression is observed.

### Example 5

### Materials and Methods

The following protocols were used to perform the experiments described in Examples 6-10.

Preparation of siRNA Cocktails with Rnase III Total RNA was Extracted from HeLa cells (RNAqueous™ Kit, Ambion) and reverse transcribed to produce cDNA (RETROscript™ Kit, Ambion). PCR primers containing T7 RNA polymerase promoters were designed to amplify a 200 bp fragment approximately 200 bp from the 5′ end of each gene of interest: human GAPDH, La, and c-fos. After PCR, the resulting templates were used in the Silencer siRNA Cocktail Kit (RNase III) to prepare siRNA cocktails to the individual genes according to the kit protocol. Briefly, the templates were used in an in vitro transcription reaction to generate dsRNA. After a brief column purification step, 15 μg of the resulting dsRNA was digested with 15 U of RNase III at 37° C. for 1 hour. The digestion products were then purified with the siRNA Purification Units included in the kit to remove any undigested dsRNA. The resulting siRNA population was quantitated using a spectrophotometer and visualized on a 20% non-denaturing acrylamide gel.

Transfections HeLa cells at 30,000 cells per well, or 293 cells at 50,000 cells per well, were grown on glass coverslips in a 24 well tissue culture plate and transfected with siRNA at the indicated concentrations using siPORT™ Lipid (Ambion).

Immunofluorescence Analysis Immunofluorescence was performed on each sample after 48 hours, using specific primary antibodies (anti-GAPDH from Ambion; anti-La from Transduction Laboratories; anti-c-FOS from Santa Cruz Biotech). A FITC-conjugated donkey anti-mouse IgG secondary antibody (Jackson Immuno Research) was used for all experiments. All samples were mounted on slides using VectaShield® with DAPI (Vector Laboratories) to allow for visualization of the cellular nuclei, and the resulting fluorescence microscopy images were digitally captured and quantified using Metamorph® software (Universal Imaging Corp.).

Size Separation of RNase III Products After a 15 minute digestion at room temperature, reaction products were separated on a 15% non-denaturing acrylamide gel. 12-15 bp region was excised and eluted in Probe Elution Buffer (Ambion) for 18 hr at 37° C., ethanol precipitated and resuspended in nuclease free water.

### Example 6

### Efficient Digestion of Distinct dsRNA Sequences

Using optimized digestion conditions the ability of RNase III to digest a number of long dsRNA substrates was analyzed. Human GAPDH, La, and c-FOS dsRNA (200 bp) was prepared by in vitro transcription (Silencer™ siRNA Cocktail Kit (RNase III); See Materials and Methods). The dsRNA was digested using 1 U RNase III per microgram of RNA for 1 hour at 37° C., to generate siRNA cocktails for each target gene. One microgram of the dsRNA before and after RNase III digestion was run on a 15% non-denaturing acrylamide gel along with a 21 bp chemically synthesized siRNA to GAPDH, which served as a size marker. The gel was stained with ethidium bromide and photographed under UV light. After a 1 hour digestion with RNase III, the long dsRNAs were reduced to fragments <30 bp, with the majority between 12-15 bp. In addition, dsRNAs to Cyclophillin, c-myc, Map Kinase 9, PKC-alpha, Raf-1, Nautilus, and h-ras made as described above, were also digested with similar results. This demonstrates the ability of the bacterial RNase III enzyme to efficiently digest a variety of dsRNA sequences.

### Example 7

### Silencing by RNase III Digested dsRNA

Next, the silencing ability of the RNase III generated siRNA cocktails was analyzed. GAPDH and La proteins in HeLa cells are abundant and endogenous levels are easily detected. However the endogenous level of c-FOS in 293 cells is relatively low, and reduction in protein levels makes the protein undetectable. In order to overcome this limitation, 293 cells were stimulated to increase c-FOS protein levels by the addition of 50 nM phorbol ester (PMA) for 24 hours prior to protein analysis. RNase III-generated siRNA cocktails to GAPDH and La were transfected into HeLa cells, and the c-fos siRNA population was transfected into 293 cells following 24 hours stimulation with 50 nM PMA. Samples were harvested at 48 hours post transfection and immunofluorescence was used to examine the gene silencing effect. The fluorescent signal from this experiment was then quantitated and normalized for cell number. Protein levels were reduced by 78% for GAPDH, 86% for La, and 75% for c-FOS by introduction of the respective siRNA cocktails. These data demonstrate that RNase III generated siRNAs are very efficient at reducing target gene expression.

### Example 8

### Silencing by 12-15 bp RNase III Digestion Products

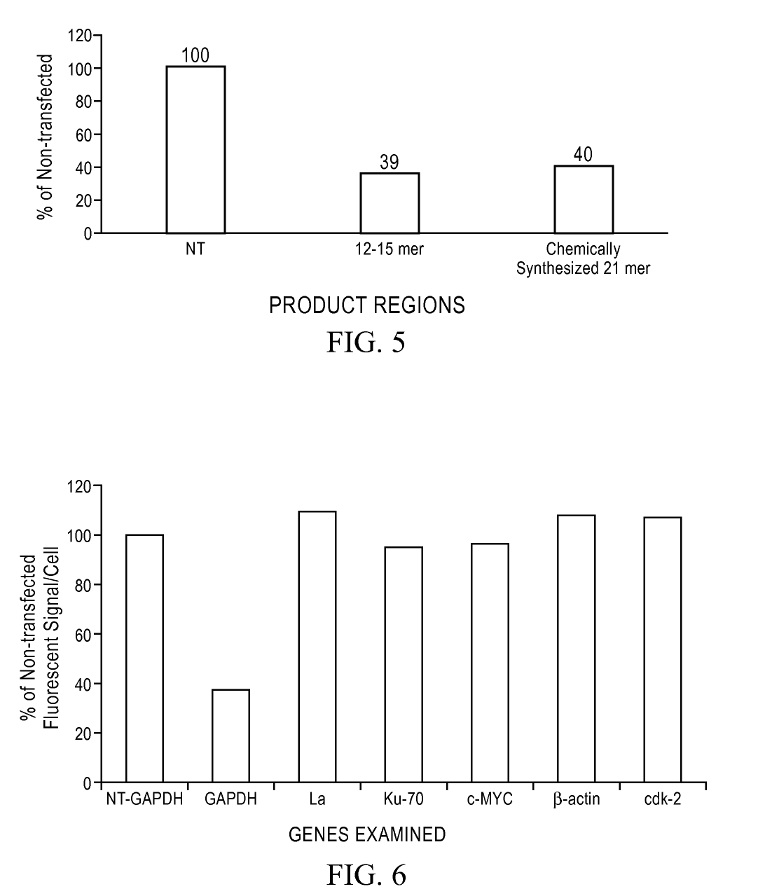

The size of chemically synthesized siRNA most often used for mediating RNAi is 21 bp (Bernstein et al., 2001). It has been shown that the 21 bp products generated by RNase III digestion are potent inhibitors of gene expression (Yang et al., 2002). However the products of a complete RNase III digestion are 12-15 bp. To compare the ability of these smaller products to reduce gene expression with 21 bp siRNA, a 200 bp GAPDH dsRNA was digested with RNase III under standard conditions and the resulting 12-15 bp fragments were acrylamide gel purified from the incomplete digestion products. HeLa cells were transfected with 100 nM final concentration of the 12-15 bp purified products, as well as with the same concentration of a 21 bp chemically synthesized siRNA known to effectively reduce GAPDH levels. 48 hours after transfection, protein levels were determined by immunofluorescence. Immunofluorescence images demonstrated reduction in GAPDH levels after transfection with the RNase III generated siRNAs. The 12-15 bp products are capable of reducing target gene expression at comparable levels to a chemically synthesized siRNA targeting GAPDH (FIG. 5). This experiment demonstrates that the smaller sized siRNA cocktails produced by RNase III reduce target gene expression upon transfection into mammalian cells and suggests that altering the digestion or purification conditions to generate longer products is unnecessary for the efficient reduction of target gene expression.

### Example 9

### Specificity of Gene Silencing

The specificity of the siRNA for reducing target gene expression was analyzed next. HeLa cells were transfected with an RNase III generated siRNA population to GAPDH, and the resulting expression levels of GAPDH and a number of nonspecific target genes (La, Ku-70, c-myc, β-actin, and cdk-2) were compared in transfected and nontransfected cells. FIG. 6 shows a 63% reduction in GAPDH levels but no detectable reduction in the other genes examined. These data suggest that nonspecific gene silencing is not occurring in cells after transfection with RNase III generated siRNA cocktails. A recent article that examined the effect of RNase III generated siRNA cocktails on related RNA binding proteins confirms the lack of nonspecific effects (Trotta et al. 2003).

### Example 10

### Comparison of RNase III Generated siRNAs to Individual Chemically Synthesized siRNAs

To compare the gene silencing effects of siRNA cocktails generated by RNase III versus individual chemically synthesized siRNAs, HeLa cells were transfected with siRNAs targeting GAPDH generated by both methods at 50 nM, 25 nM and 12.5 nM final concentration. The resulting protein levels were examined 48 hours after transfection. siRNAs prepared by both methods efficiently reduced GAPDH protein levels in a dose dependent manner, although higher concentrations of RNase III-generated siRNAs were required to maximally reduce GAPDH expression levels.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
U.S. Prov. Appl. 60/353,332
U.S. Pat. RE 35,443
U.S. Pat. No. 4,554,101
U.S. Pat. No. 4,659,774
U.S. Pat. No. 4,682,195
U.S. Pat. No. 4,683,202
U.S. Pat. No. 4,684,611
U.S. Pat. No. 4,704,362
U.S. Pat. No. 4,704,362
U.S. Pat. No. 4,786,600
U.S. Pat. No. 4,800,159
U.S. Pat. No. 4,816,571
U.S. Pat. No. 4,883,750
U.S. Pat. No. 4,952,496
U.S. Pat. No. 4,952,500
U.S. Pat. No. 4,959,463
U.S. Pat. No. 5,026,645
U.S. Pat. No. 5,037,745
U.S. Pat. No. 5,102,802
U.S. Pat. No. 5,141,813
U.S. Pat. No. 5,214,136
U.S. Pat. No. 5,221,619
U.S. Pat. No. 5,223,618
U.S. Pat. No. 5,264,566
U.S. Pat. No. 5,302,523
U.S. Pat. No. 5,322,783
U.S. Pat. No. 5,378,825
U.S. Pat. No. 5,384,253
U.S. Pat. No. 5,428,148
U.S. Pat. No. 5,445,934
U.S. Pat. No. 5,446,137
U.S. Pat. No. 5,464,765
U.S. Pat. No. 5,466,786
U.S. Pat. No. 5,470,967
U.S. Pat. No. 5,480,980
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,583,013
U.S. Pat. No. 5,589,466
U.S. Pat. No. 5,591,601
U.S. Pat. No. 5,591,616
U.S. Pat. No. 5,602,240
U.S. Pat. No. 5,602,244
U.S. Pat. No. 5,610,042
U.S. Pat. No. 5,610,289
U.S. Pat. No. 5,614,617
U.S. Pat. No. 5,623,070
U.S. Pat. No. 5,637,683
U.S. Pat. No. 5,645,897
U.S. Pat. No. 5,652,099
U.S. Pat. No. 5,656,610
U.S. Pat. No. 5,670,663
U.S. Pat. No. 5,672,697
U.S. Pat. No. 5,681,947
U.S. Pat. No. 5,700,922
U.S. Pat. No. 5,702,932
U.S. Pat. No. 5,705,629
U.S. Pat. No. 5,708,154
U.S. Pat. No. 5,714,606
U.S. Pat. No. 5,728,525
U.S. Pat. No. 5,736,524
U.S. Pat. No. 5,744,305
U.S. Pat. No. 5,763,167
U.S. Pat. No. 5,777,092
U.S. Pat. No. 5,780,448
U.S. Pat. No. 5,789,215
U.S. Pat. No. 5,792,847
U.S. Pat. No. 5,795,715
U.S. Pat. No. 5,824,528
U.S. Pat. No. 5,843,650
U.S. Pat. No. 5,846,709
U.S. Pat. No. 5,846,783
U.S. Pat. No. 5,849,497
U.S. Pat. No. 5,849,546
U.S. Pat. No. 5,849,547
U.S. Pat. No. 5,858,652
U.S. Pat. No. 5,858,988
U.S. Pat. No. 5,859,221
U.S. Pat. No. 5,866,366
U.S. Pat. No. 5,869,320
U.S. Pat. No. 5,872,232
U.S. Pat. No. 5,882,864
U.S. Pat. No. 5,886,165
U.S. Pat. No. 5,891,681
U.S. Pat. No. 5,891,681
U.S. Pat. No. 5,912,148
U.S. Pat. No. 5,916,776
U.S. Pat. No. 5,916,779
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,624
U.S. Pat. No. 6,114,152
U.S. Pat. No. 6,251,666
U.S. Pat. No. 6,309,823
U.S. Pat. No. 6,316,193
U.S. Pat. No. 6,322,971
U.S. Pat. No. 6,324,479
U.S. Pat. No. 6,329,140
U.S. Pat. No. 6,329,209
PCT Applic. No. PCT/US87/00880
PCT Applic. No. PCT/US89/01025
PCT Applic. No. WO 00/44914
PCT Applic. No. WO 01/68836
PCT Applic. No. WO 01/36646
PCT Applic. No. WO 89/100700
PCT Applic. No. WO 99/32619
PCT Applic. No. WO 88/10315
PCT Applic. No. WO 94/09699
PCT Applic. No. WO 90/07641
PCT Applic. No. WO 95/06128
PCT Applic. No. WO 91/05,866
PCT Applic. No. WO 91/02,818
GB Application No. 2 202 328
European Appl. 0,178,863
European Appl. 266,032
European Appl. 266,032
European Appl. 320,308
European Appl. 329,822
Antisense Drug Technology, Stanley T. Crooke (ed), Marcel Dekker and Co, Basel, Switzerland, Chapter 6, 2001.
Ausubel et al., In: Current Protocols in Molecular Biology, John, Wiley and Sons, Inc, New York, 1994.
Baglioni and Nilson, Interferon, 5:23-42, 1983.
Beaucage, and Lyer, Tetrahedron, 48:2223-2311, 1992.
Bernstein et al., Nature, 409: 363-366, 2001.
Blaszczyk et al., Structure, 9(12):1225-1236, 2001.
Bosher and Labouesse, Nat. Cell Biol., 2:E31-E36, 2000.
Brown et al., Ambion TechNotes 9(1): 3-6, 2002.
Brummelkamp et al., Science, 296(5567):550-553, 2002.
Calegari et al., Proc Natl Acad Sci USA, 99(22):14236, 2002.
Caplen et al., Proc Natl Acad Sci USA, 98: 9742-9747, 2001.
Chen and Okayama, Mol. Cell. Biol., 7(8):2745-2752, 1987.
Cogoni and Macino, Science, 286:342-2344, 1999.
Cogoni. and Macino, Nature 399:166-169, 1999.
Court, in “Control of Messenger RNA Stability” (J. G. Belarco and G. Brauerman, eds., Academic Press, New York) 1993.
Cummins et al., In IRT: Nucleosides and nucleosides, La Jolla Calif., 72, 1996.
Dalmay et al. EMBO J., 20:2069-2078, 2001.
Dalmay et al., Cell, 101:543-553, 2000.
DeRisi et al., Nature Genetics 14:457-460, 1996.
Didenko, Biotechniques, 31(5):1106-16, 1118, 1120-1, 2001.
Dunn, in “The Enzymes” (P. D. Boyer, ed., Academic Press, New York) 1982.
Elbashir et al., Genes Dev. 15: 188-200, 2001.
Elbashir et al., Nature, 411:494-498, 2001.
Emptage et al., Neuron, 2001 January; 29(1):197-208, 2001.
Fechheimer et al., Proc. Natl. Acad. Sci. USA, 84:8463-8467, 1987.
Fire et al., Nature, 391:806-811, 1998.
Fodor et al., Science, 251:767-773, 1991.
Forster et al. Nucleic Acids Res., 13(3):745-761, 1985.
Fraley et al., Proc. Natl. Acad. Sci. USA, 76:3348-3352, 1979.
Froehler et al., Nucleic Acids Res., 14(13):5399-5407, 1986.
Frohman, In: PCR Protocols: A Guide To Methods and Applications, Academic Press, NY, 1990.
Gillam et al., J. Biol. Chem., 253:2532, 1978.
Gillam et al., Nucleic Acids Res., 6:2973, 1979.
Gopal, Mol. Cell. Biol., 5:1188-1190, 1985.
Graham and Van Der Eb, Virology, 52:456-467, 1973.
Griffey et al., J. Mass. Spectrom., 32(3):305-13, 1997.
Grishok et al., Cell, 106: 23-34, 2001.
Hacia et al., Nature Genetics, 14:441-447, 1996.
Hamilton and Baulcombe, Science, 286:950-952, 1999.
Hammond et al., Nat. Rev. Genet., 2(2):110-9, 2001.
Harland and Weintraub, J. Cell. Biol., 101:1094-1099, 1985.
Higgins et al., Comput. Appl. Biosci., 8(2):189-191, 1992.
Hutvagner et al., Science, 293:834-838, 2001.
Innis et al., Proc. Natl. Acad. Sci. USA, 85(24):9436-9440, 1988.
Inouye and Inouye, Nucleic Acids Res., 13:3101-3109, 1985.
Itakura and Riggs, Science, 209:1401-1405, 1980.
Itakura et al., J. Biol. Chem., 250:4592 1975.
Jacque et al., Nature, 2002.
Johnson et al., In: Biotechnology and Pharmacy, Pezzuto et al., (eds), Chapman and Hall, New York, 1993.
Kaeppler et al., Plant Cell Reports, 9:415-418, 1990.
Kaneda et al., Science, 243:375-378, 1989.
Kato et al, J. Biol. Chem., 266:3361-3364, 1991.
Ketting et al., Cell, 99:133-141, 1999.
Kharrat et al., EMBO. J., 14(14):3572-3584, 1995.
Khorana, Science, 203, 614 1979.
Klostermeier and Millar, Biopolymers, 61(3):159-79, 2001-2002
Knight et al., Science, 2:2, 2001.
Kornberg and Baker, In: DNA Replication, 2nd Ed., Freeman, San Francisco, 1992.
Kwoh et al., Proc. Natl. Acad. Sci. USA, 86: 1173, 1989.
Kyte and Doolittle, J. Mol. Biol., 157(1):105-132, 1982.
Lee et al., DNA Cell Biol., 16(11):1267-1275, 1997.
Lin and Avery, Nature, 402:128-129, 1999.
Maskos et al., Nuc. Acids. Res. 20:1679-1684, 1992.
Miyagishi and Taira, Biotechnol., 5:497-500, 2002.
Montgomery et al., Proc. Natl. Acad. Sci. USA, 95:155-2-15507, 1998.
Mourrain et al., Cell, 101:533, 2000.
Nicholson, FEMS Microbiol. Rev. 23: 371, 1999.
Nicolau and Sene, Biochim. Biophys. Acta, 721:185-190, 1982.
Nicolau et al., Methods Enzymol., 149:157-176, 1987.
Nishikura, Cell 16:415-418, 2001.
Novina et al., Nat. Med., 8:681-686, 2002.
Ohara et al., Proc. Natl. Acad. Sci. USA, 86:5673-5677, 1989.
Omirulleh et al., Plant Mol. Biol., 21(3):415-428, 1993.
Paul et al., Nat. Biotechnol., 20:505-508, 2002.
Pease et al., Proc. Natl. Acad. Sci. USA, 91:5022-5026, 1994.
Plasterk and Ketting, Curr. Opin. Genet. Dev., 10:562-567, 2000.
Potrykus et al., Mol. Gen. Genet., 199(2):169-77, 1985.
Rippe et al., Mol. Cell. Biol., 10:689-695, 1990.
Robertson et al., J. Biol. Chem. 243: 82, 1968.
Saiki et al. Science, 230:1350-1354, 1985
Sambrook et al., In: Molecular cloning, Cold Spring Harbor Laboratory Press, Cold Spring Harbor, N.Y., 2001.
Scheit, In: Synthesis and Biological Function, Wiley-Interscience, NY, 171-172, 1980.
Schena et al., Science 270:467-470, 1995a.
Schena et al., Proc. Natl. Acad. Sci. USA 93:10539-11286, 1995b.
Shalon et al., Genome Res. 6:639-645, 1996.
Sharp and Zamore, Science, 287:2431-2433, 2000.
Sharp, Genes Dev., 13:139-141, 1999.
Shoemaker et al., Nature Genetics, 14:450-456, 1996.
Smardon et al., Curr. Biol., 10:169-178, 2000.
Sui et al., Proc. Natl. Acad. Sci. USA, 99(8):5515-5520, 2002.
Tabara et al., Cell, 99:123-132, 1999.
Thompson et al., Nucleic Acids Res., 22(22):4673-4680, 1994.
Trotta et al., Cancer Cell 3:(2):145-60, 2003.
Tuschl, Chembiochem., 2:239-245, 2001.
Walker et al., Proc. Natl. Acad. Sci. USA, 89:392-396 1992.
Waterhouse et al., Nature, 411:834-842, 2001.
Williams et al., Int. J. Dev. Biol., 41(2):359-364, 1997.
Wong et al., Gene, 10:87-94, 1980.
Wu-Scharf et al., Science, 290:1159-1162, 2000.
Yang et al., Proc. Natl. Acad. Sci. USA, 99(15): 9942-7, 2002.
Yu et al., Proc. Natl. Acad. Sci. USA, 99:6047-6052, 2002.
Zamore et al., Cell, 101:25-33, 2000.
Zamore, Nat. Struct. Biol., 8:746-750, 2001.

1.-9. (canceled)

10. A method for achieving RNA interference of a target gene in a cell using one or more siRNA molecules comprising:
a) generating at least one double-stranded DNA template corresponding to part of the target gene, wherein the DNA template comprises an SP6, T3, or T7 promoter on at least one strand;
b) transcribing the template, wherein either i) a single RNA strand with a complementarity region, or ii) first and second complementary RNA strands is/are created;
c) hybridizing either the single complementary RNA strand or first and second complementary RNA strands to create a dsRNA molecule corresponding to the target gene;
d) incubating the dsRNA molecule with a polypeptide comprising an RNase III domain, under conditions to allow cleavage of the dsRNA into at least two siRNA; and
e) transfecting at least one siRNA into the cell.

11. The method of claim 10, wherein the polypeptide is RNase III.

12. The method of claim 10, wherein the polypeptide is chimeric.

13. The method of claim 10, wherein multiple siRNA molecules are transfected into the cell.

14. A kit for generating siRNA molecules comprising:
a) recombinant, prokaryotic RNase III;
b) RNase III buffer; and
c) a control nucleic acid.

15. The kit of claim 14, wherein the RNase III is in an enzyme dilution buffer.

16. The kit of claim 14, further comprising an SP6, T3 or T7 RNA polymerase.

17. The kit of claim 16, wherein the polymerase is in an enzyme mix comprising inorganic pyrophosphatase, at least one RNase inhibitor, and about 1% CHAPS.

18. The kit of claim 16, further comprising an SP6, T3, or T7 polymerase buffer.

19. The kit of claim 16, further comprising ATP, CTP, GTP, and UTP.

20. The kit of claim 14, wherein the RNase III buffer comprises Tris and a salt.

21. The kit of claim 14, wherein the control nucleic acid is DNA and comprises an SP6, T3, or T7 promoter.

22. The kit of claim 14, wherein the control nucleic acid is dsRNA.

23. The kit of claim 14, wherein the control nucleic acid is a DNA template capable of being transcribed into a dsRNA.

24. The kit of claim 16, further comprising RNase A.

25. The kit of claim 14, further comprising a cartridge, column, or filter for isolating nucleic acids.

26. The kit of claim 25, further comprising binding buffer comprising NaCl.

27. The kit of claim 25, further comprising wash buffer comprising NaCl.

28. The kit of claim 25, further comprising an elution solution comprising Tris and EDTA.

29-37. (canceled)
